(19) United States
(12) Patent Application Publication
(10) Pub. No.: US 2023/0124417 A1
(43) Pub. Date: Apr. 20, 2023

(54) COMPUTER-IMPLEMENTED METHODS FOR QUANTITATION OF FEATURES OF INTEREST IN WHOLE-SLIDE IMAGING

(71) Applicant: Boundless Bio, Inc., San Diego, CA (US)

(72) Inventors: Nam-Phuong NGUYEN, San Diego, CA (US); Eva Lorena MORA-BLANCO, San Diego, CA (US); Kristen TURNER, San Diego, CA (US); Julie WIESE, San Diego, CA (US); Jason CHRISTIANSEN, San Diego, CA (US); Mihir BAFNA, San Diego, CA (US)

(21) Appl. No.: 17/906,206

(22) PCT Filed: Mar. 15, 2021
(86) PCT No.: PCT/US2021/022308

§ 371 Date: Sep. 13, 2022

Related U.S. Application Data
(60) Provisional application No. 62/990,188, filed on Mar. 16, 2020.

(51) Int. Cl. G16B 25/00; G16B 40/20; G06V 20/69; G06T 3/40

(52) U.S. Cl. CPC G16B 25/00; G06T 3/40; G06V 20/695; G06V 20/698; G16B 40/20

(57) ABSTRACT

Methods and systems for quantitation of features of interest (e.g., extrachromosomal DNA) in whole slide images are disclosed herein. One or more methods and systems described herein are used to reduce bias in the quantitation of the features of interest.

### CROSS REFERENCE

This application claims the benefit of U.S. Provisional Pat. Application No. 62/990,188, filed Mar. 16, 2020, which is incorporated by reference herein in its entirety for all intents and purposes.

### BACKGROUND

Imaging of cells provides useful information for understanding biological mechanisms, pathologies, and effects of treatments. In images of cells, quantitation of features of interest, e.g., nucleic acid molecules, proteins, or macromolecules provides mechanistic insights for biological processes or pathologies. However, in some instances, the identification and analysis of features of interest introduce bias, are laborious, have low resolution, or have low throughput. Thus, new technologies to address these issues are necessary.

### SUMMARY

Recognized herein is a need for improved methods and systems for high-throughput quantitation or quantification of features of interest in an image or plurality of images (e.g., a microscope slide).

Disclosed herein, in certain embodiments, are methods and systems for reducing or eliminating bias in the quantitation of features of interest present in a cell or plurality of cells in an image that is performed in a high-throughput format. In some embodiments, the methods and systems disclosed herein use automated, computer-implemented methods for quantitation and quantification of the features, thereby obviating the need for manual identification of features of interest and reducing bias. In some embodiments, the features of interest comprise nucleic acid molecules, e.g., deoxyribonucleic acid (DNA). In certain embodiments, the features of interest include extrachromosomal DNA (ecDNA). In certain embodiments, the features of interest include circular ecDNA.

In an aspect, provided herein is a computer-implemented method of eliminating bias in detecting nucleic acids present in a plurality of cells in a first image, the computer-implemented method comprising: (a) down-sampling, by at least one processor, the first image, thereby generating a down-sampled image; (b) segmenting, by the at least one processor, the down-sampled image, wherein the segmenting comprises removing, from the down-sampled image, one or more compact nuclei originating from the plurality of cells, thereby generating a compact-nuclei-free image; (c) automatically identifying, by the at least one processor, a plurality of first regions in the compact-nuclei-free image, wherein each region of the plurality of first regions has a summed pixel intensity value above a threshold intensity value; (d) generating, by the at least one processor, a plurality of contours around at least a subset of the plurality of first regions in the compact-nuclei-free image; (e) partitioning, by the at least one processor, the first image into a plurality of second images, using pixel locations of the plurality of contours, wherein each image of the plurality of second images comprises a single region corresponding to a single contour of the plurality of contours of the compact-nuclei-free image; (f) segmenting, by the at least one processor, each image of the plurality of second images to identify one or more nucleic acid features; and (g) electronically outputting information indicative of the presence or quantity of the one or more nucleic acid features present in the plurality of cells in the first image.

In some embodiments, the one or more nucleic acid features comprises extrachromosomal deoxyribonucleic acid (ecDNA). In some embodiments, the one or more nucleic acid features comprises a chromosomal homogenous staining region (HSR). In some embodiments, the one or more nucleic acid features comprises one or more gene amplifications. In some embodiments, the one or more nucleic acid features comprises nuclei in metaphase. In some embodiments, the information of (g) comprises one or more members selected from the group consisting of a quantity of ecDNA, a number of cells containing ecDNA, and a percentage of cells containing ecDNA. In some embodiments, the information of (g) comprises a quantity of HSR on a chromosome, a quantity of HSR on ecDNA, or a ratio of pixel intensity of FISH on HSR on chromosomes to pixel intensity of FISH on ecDNA. In some embodiments, the down-sampling in (a) comprises reducing a resolution of the first image or shrinking dimensions of the first image by a percentage. In some embodiments, the percentage is between about 70% and about 95%. In some embodiments, the segmenting in (b) comprises white top-hat filtering. In some embodiments, the white top-hat filtering comprises a morphological opening, wherein the morphological opening comprises performing, using the at least one processor, one or more erosions, dilations, or a combination thereof. In some embodiments, (b) comprises removing pixels belonging to the morphological opening. In some embodiments, the one or more compact nuclei comprises a non-metaphase nucleus. In some embodiments, (c) comprises sliding a window across the compact-nuclei-free image, wherein at each pixel location of the compact-nucleic-free image, a summation of pixel intensities in the window is performed. In some embodiments, the plurality of first regions is generated from the window only if the summation of pixel intensities is greater than the threshold intensity value. In some embodiments, the window has a kernel size of 16 pixels by 16 pixels. In some embodiments, the pixel locations of (e) are image coordinates of centroids of the plurality of contours. In some embodiments, an image of the plurality of second images comprises a single metaphase nucleus. In some embodiments, the single metaphase nucleus is located in a center of the image. In some embodiments, the plurality of contours comprises or surrounds overlapping first regions of the plurality of first regions. In some embodiments, the one or more nucleic acid features comprises ecDNA, wherein the ecDNA comprises a first labeled probe and a second labeled probe, wherein the first and the second labeled probes each hybridize to a different feature. In some embodiments, the different feature comprises a gene-specific sequence. In some embodiments, the computer-implemented method further comprises separately quantifying the ecDNA comprising the first labeled probe and the ecDNA comprising the second labeled probe. In some embodiments, each contour of the plurality of contours corresponds to a cell of the plurality of cells. In some embodiments, the first image comprises a plurality of images of a microscope slide comprising the plurality of cells. In some embodiments, the computer-implemented method further comprises, prior to (a), overlapping, by the at least one processor, the plurality of images to generate the first image. In some embodiments, wherein the plurality of images comprises at least 20 images. In some embodiments, the one or more nucleic acid features comprises ecDNA, wherein the ecDNA comprises labeled probes. In some embodiments, the labeled probes comprises gene-specific fluorescence in situ hybridization (FISH) probes. In some embodiments, the labeled probes comprise colorimetric in situ hybridization (CISH) probes. In some embodiments, the first image further comprises an additional plurality of cells that do not have ecDNA. In some embodiments, the computer-implemented method further comprises performing a statistical operation on the nucleic acid features identified in (f). In some embodiments, the statistical operation compares a pixel intensity and location of the nucleic acid features to a pixel intensity and location of an additional set of features of interest. In some embodiments, the additional set of features of interest comprises chromosomal DNA. In some embodiments, the statistical operation uses the comparison to remove outliers. In some embodiments, (d) comprises using a statistical clustering algorithm of the summed pixel intensity value to generate the plurality of contours. In some embodiments, (f) further comprises quantifying the one or more nucleic acid features. In some embodiments, (f) further comprises enumerating the one or more nucleic acid features.

In another aspect, disclosed herein is a computer-implemented system for performing non-biased, automatic detection of nucleic acids present in a plurality of cells in a first image, comprising: at least one processor configured to perform executable instructions and a memory comprising the executable instructions, which, when executed by the at least one processor, causes the at least one processor to: (a) down-sample the first image, thereby generating a down-sampled image; (b) segment the down-sampled image, wherein the segmenting comprises removing, from the down-sampled image, one or more compact nuclei originating from the plurality of cells, thereby generating a compact-nuclei-free image;(c) automatically identify a plurality of first regions in the compact-nuclei-free image, wherein each region of the plurality of first regions has a summed pixel intensity value above a threshold intensity value; (d) generate a plurality of contours around at least a subset of the plurality of first regions in the compact-nuclei-free image; (e) partition the first image into a plurality of second images, using pixel locations of the plurality of contours, wherein each image of the plurality of second images comprises a single region corresponding to a single contour of the plurality of contours of the compact-nuclei-free image; (f) segment each image of the plurality of second images to identify one or more nucleic acid features; and (g) electronically output information indicative of the presence or quantity of the one or more nucleic acid features present in the plurality of cells in the first image.

In some embodiments, the one or more nucleic acid features comprises extrachromosomal deoxyribonucleic acid (ecDNA). In some embodiments, the one or more nucleic acid features comprises a chromosomal homogenous staining region (HSR). In some embodiments, the one or more nucleic acid features comprises one or more gene amplifications. In some embodiments, the one or more nucleic acid features comprises nuclei in metaphase. In some embodiments, the information of (g) comprises one or more members selected from the group consisting of a quantity of ecDNA, a number of cells containing ecDNA, and a percentage of cells containing ecDNA. In some embodiments, the information of (g) comprises a quantity of HSR on a chromosome, a quantity of HSR on ecDNA, or a ratio of pixel intensity of FISH on HSR on chromosomes to pixel intensity of FISH on ecDNA.

In another aspect, provided herein is a non-transitory computer readable storage medium encoded with a computer program including instructions executable by a processor to perform non-biased, automatic detection of nucleic acids present in a plurality of cells in a first image, the computer program comprising: (a) a software module for down-sampling the first image, thereby generating a down-sampled image; (b) a software module for segmenting the down-sampled image, wherein the segmenting comprises removing, from the down-sampled image, one or more compact nuclei originating from the plurality of cells, thereby generating a compact-nuclei-free image; (c) a software module for automatically identifying a plurality of first regions in the compact-nuclei-free image, wherein each region of the plurality of first regions has a summed pixel intensity value above a threshold intensity value; (d) a software module for generating a plurality of contours around at least a subset of the plurality of first regions in the compact-nuclei-free image; (e) a software module for partitioning the first image into a plurality of second images, using pixel locations of the plurality of contours, wherein each image of the plurality of second images comprises a single region corresponding to a single contour of the plurality of contours of the compact-nuclei-free image; (f) a software module for segmenting each image of the plurality of second images to identify one or more nucleic acid features; and (g) a software module for electronically outputting information indicative of the presence or quantity of the one or more nucleic acid features present in the plurality of cells in the first image.

In some embodiments, the one or more nucleic acid features comprises extrachromosomal deoxyribonucleic acid (ecDNA). In some embodiments, the one or more nucleic acid features comprises a chromosomal homogenous staining region (HSR). In some embodiments, the one or more nucleic acid features comprises one or more gene amplifications. In some embodiments, the one or more nucleic acid features comprises nuclei in metaphase. In some embodiments, the information of (g) comprises one or more members selected from the group consisting of a quantity of ecDNA, a number of cells containing ecDNA, and a percentage of cells containing ecDNA. In some embodiments, the information of (g) comprises a quantity of HSR on a chromosome, a quantity of HSR on ecDNA, or a ratio of pixel intensity of FISH on HSR on chromosomes to pixel intensity of FISH on ecDNA.

In another aspect, provided herein is a computer-implemented method of eliminating bias in a quantification of features of interest present in a plurality of cells in an image, the computer-implemented method comprising: (a) partitioning, by at least one processor, the image into a plurality of first regions; (b) segmenting, by the at least one processor, each region of the plurality of first regions to identify a first set of features, wherein the segmenting is performed using at least one pixel intensity value relative to a background intensity value; (c) automatically identifying, by the at least one processor, boundaries of the plurality of cells across the plurality of first regions using the at least one pixel intensity value and a pixel location of at least one feature of the first set of features; (d) generating, by the at least one processor, a plurality of second regions using the boundaries of the plurality of cells; (e) segmenting, by the at least one processor, each region of the plurality of second regions to identify the features of interest and quantify the features of interest present in a cell of the plurality of cells; and (f) electronically outputting a report indicative of a quantity of the features of interest present in the cell.

In some embodiments, the image comprises a plurality of images of a microscope slide comprising the plurality of cells. In some embodiments, the method further comprises, prior to (a), overlapping, by the at least one processor, the plurality of images to generate the image. In some embodiments, the plurality of images comprises at least 20 images. In some embodiments, the first set of features or the features of interest comprises non-chromosomal DNA. In some embodiments, the non-chromosomal DNA is extrachromosomal DNA (ecDNA). In some embodiments, the first set of features or the features of interest further comprises chromosomal DNA. In some embodiments, the first set of features or the features of interest further comprises fluorescently labeled probes. In some embodiments, the fluorescently labeled probes comprises gene-specific fluorescence in situ hybridization (FISH) probes. In some embodiments, the first set of features or the features of interest comprise gene-specific labeled probes. In some embodiments, the labeled probes comprise FISH probes or colorimetric in situ hybridization (CISH) probes. In some embodiments, the image further comprises an additional plurality of cells that do not comprise the features of interest. In some embodiments, the method further comprises performing a statistical operation on the features of interest identified in (e). In some embodiments, the statistical operation compares a pixel intensity and location of a subset of the features of interest to a pixel intensity and location of an additional subset of the features of interest. In some embodiments, the subset of the features of interest comprises ecDNA and the additional subset of the features of interest comprises chromosomal DNA. In some embodiments, the statistical operation uses the comparison to remove outliers. In some embodiments, the cell is a metaphase cell. In some embodiments, the cell is an interphase cell. In some embodiments, (c) comprises using a statistical clustering algorithm of the at least one pixel intensity value to identify the boundaries of the plurality of cells. In some embodiments, the statistical clustering algorithm is density-based spatial clustering of applications with noise (DBSCAN). In some embodiments, (d) comprises overlapping a cluster of the boundaries of the plurality of cells to generate the plurality of second regions. In some embodiments, each of the plurality of second regions has a single cell.

In another aspect, disclosed herein is a computer-implemented system for performing non-biased, automatic quantification of features of interest present in a plurality of cells in an image, comprising: at least one processor configured to perform executable instructions and a memory comprising the executable instructions, which, when executed by the at least one processor, causes the at least one processor to: (a) partition the image into a plurality of first regions; (b) segment each of the plurality of first regions to identify a first set of features, wherein the segmenting is performed using at least one pixel intensity value relative to a background intensity value; (c) identify boundaries of the plurality of cells across the plurality of first regions using the at least one pixel intensity value and a pixel location of at least one feature of the first set of features; (d) generate a plurality of second regions using the boundaries of the plurality of cells; (e) segment each of the plurality of second regions to identify the features of interest and quantify the features of interest present in a cell of the plurality of cells; and (f) electronically output a report indicative of a quantity of the features of interest present in the cell.

In some embodiments, the image comprises a plurality of images of a microscope slide comprising the plurality of cells. In some embodiments, the executable instructions cause the at least one processor to, prior to (a), overlap the plurality of images to generate the image. In some embodiments, the plurality of images comprises at least 20 images. In some embodiments, the first set of features or the features of interest comprises non-chromosomal DNA. In some embodiments, the non-chromosomal DNA is extrachromosomal DNA (ecDNA). In some embodiments, the first set of features or the features of interest further comprises chromosomal DNA. In some embodiments, the first set of features or the features of interest further comprises fluorescently labeled probes. In some embodiments, the fluorescently labeled probes comprises gene-specific fluorescence in situ hybridization (FISH) probes. In some embodiments, the first set of features or the features of interest comprise gene-specific labeled probes. In some embodiments, the labeled probes comprise FISH probes or colorimetric in situ hybridization (CISH) probes. In some embodiments, the image further comprises an additional plurality of cells that do not comprise the features of interest. In some embodiments, the executable instructions cause the at least one processor to perform a statistical operation on the features of interest identified in (e). In some embodiments, the statistical operation compares a pixel intensity and location of a subset of the features of interest to a pixel intensity and location of an additional subset of the features of interest. In some embodiments, the subset of the features of interest comprises ecDNA and the additional subset of the features of interest comprises chromosomal DNA. In some embodiments, the statistical operation uses the comparison to remove outliers. In some embodiments, the cell is a metaphase cell. In some embodiments, the cell is an interphase cell. In some embodiments, (c) comprises using a statistical clustering of the at least one pixel intensity value to identify the boundaries of the plurality of cells. In some embodiments, the statistical clustering is density-based spatial clustering of applications with noise (DBSCAN). In some embodiments, (d) comprises overlapping a cluster of the boundaries of the plurality of cells to generate the plurality of second regions. In some embodiments, each of the plurality of second regions has a single cell.

In yet another aspect, disclosed herein is a non-transitory computer readable storage medium encoded with a computer program including instructions executable by a processor to perform non-biased, automatic quantification of features of interest present in a plurality of cells in an image, the computer program comprising: (a) a software module for partitioning the image into a plurality of first regions; (b) a software module for segmenting each of the plurality of first regions to identify a first set of features, wherein the segmenting is performed using at least one pixel intensity value relative to a background intensity value; (c) a software module for automatically identifying boundaries of the plurality of cells across the plurality of first regions using the at least one pixel intensity value and a pixel location of at least one feature of the first set of features; (d) a software module for generating a plurality of second regions using the boundaries of the plurality of cells; (e) a software module for segmenting each of the plurality of second regions to identify the features of interest and quantify the features of interest present in a cell of the plurality of cells; and (f) a software module for electronically outputting a report indicative of a quantity of the features of interest present in the cell.

In some embodiments, the image comprises a plurality of images of a microscope slide comprising the plurality of cells. In some embodiments, the computer program further comprises a software module for overlapping the plurality of images to generate the image. In some embodiments, the plurality of images comprises at least 20 images. In some embodiments, the first set of features or the features of interest comprises non-chromosomal DNA. In some embodiments, the non-chromosomal DNA is extrachromosomal DNA (ecDNA). In some embodiments, the first set of features or the features of interest further comprises chromosomal DNA. In some embodiments, the first set of features or the features of interest further comprises fluorescently labeled probes. In some embodiments, the fluorescently labeled probes comprises gene-specific fluorescence in situ hybridization (FISH) probes. In some embodiments, the first set of features or the features of interest comprise gene-specific labeled probes. In some embodiments, the labeled probes comprise FISH probes or colorimetric in situ hybridization (CISH) probes. In some embodiments, the image further comprises an additional plurality of cells that do not comprise the features of interest. In some embodiments, the computer program further comprises a software module for performing a statistical operation on the features of interest identified in (e). In some embodiments, the statistical operation compares a pixel intensity and location of a subset of the features of interest to a pixel intensity and location of an additional subset of the features of interest. In some embodiments, the subset of the features of interest comprises ecDNA and the additional subset of the features of interest comprises chromosomal DNA. In some embodiments, the statistical operation uses the comparison to remove outliers. In some embodiments, cell is a metaphase cell. In some embodiments, the cell is an interphase cell. In some embodiments, the software module in (c) uses a statistical clustering of the at least one pixel intensity value to identify the boundaries of the plurality of cells. In some embodiments, the statistical clustering is density-based spatial clustering of applications with noise (DBSCAN). In some embodiments, the software module in (d) overlaps a cluster of the boundaries of the plurality of cells to generate the plurality of second regions. In some embodiments, each of the plurality of second regions has a single cell.

In yet another aspect, provided herein is a computer-implemented method of eliminating bias in a quantification of features of interest present in a plurality of cells in an image, the computer-implemented method comprising: (a) partitioning, by at least one processor, the image into a plurality of first regions; (b) segmenting, by the at least one processor, each of the plurality of first regions to identify a first set of features, wherein the segmenting is performed using at least one pixel intensity value relative to a background intensity value; (c) automatically identifying, by the at least one processor, boundaries of the plurality of cells using the at least one pixel intensity value and a pixel location of at least one feature of the first set of features across the plurality of first regions; (d) segmenting, by the at least one processor, a plurality of second regions defined by the boundaries to identify the features of interest and quantify the features of interest present in a cell of the plurality of cells; and (e) electronically outputting a report indicative of a quantity of the features of interest present in the cell.

Another aspect of the present disclosure provides a non-transitory computer-readable medium comprising machine-executable code that, upon execution by one or more computer processors, implements any of the methods above or elsewhere herein.

Another aspect of the present disclosure provides a system comprising one or more computer processors and computer memory coupled thereto. The computer memory comprises machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and descriptions are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present subject matter will be obtained by reference to the following detailed description that sets forth illustrative embodiments and the accompanying drawings (also “Figure” and “FIG.”) of which:

FIG. 1A shows a non-limiting example of a workflow of the methods and processes described herein. FIG. 1B shows another non-limiting example of a workflow of the methods and processes described herein.

FIG. 2 shows a non-limiting example of a computing device; in this case, a device with one or more processors, memory, storage, and a network interface.

FIG. 3 shows a non-limiting example of a web/mobile application provision system; in this case, a system providing browser-based and/or native mobile user interfaces.

FIG. 4 shows a non-limiting example of a cloud-based web/mobile application provision system; in this case, a system comprising an elastically load-balanced, auto-scaling web server and application server resources as well synchronously replicated databases.

FIG. 5 shows a non-limiting example workflow of the methods and processes described herein.

FIG. 6 shows an example image of a whole slide comprising cells.

FIG. 7 shows an example image of a partitioned image.

FIG. 8 shows an example of a segmented image containing features of interest.

FIG. 9 shows an example image of labeled boundaries of cells comprising a feature of interest.

FIG. 10 shows an example of a set of regions generated using the boundaries of cells labeled in a method disclosed herein.

FIG. 11 shows an example of outliers that are removed using a process disclosed herein.

FIG. 12 shows an example of a downsampled image.

FIG. 13 shows an example of a segmented image with compact nuclei removed.

FIG. 14 shows an example of contours generated from a plurality of first regions with at least a threshold summed pixel intensity value.

FIG. 15 shows an example of partitioned regions of an image using a process disclosed herein.

FIG. 16 shows an example of a partitioned region generated using a process disclosed herein.

FIG. 17 shows example data comparing a manual image processing method with an automated process described herein.

FIG. 18 shows example data of fluorescence in situ hybridization (FISH) obtained using a process described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Unless otherwise defined, all technical terms used herein have the same meaning as commonly understood by one of ordinary skill in the art to which the present subject matter belongs. As used in this specification and the appended claims, the singular forms “a,” “an,” and “the” include plural references unless the context clearly dictates otherwise. Any reference to “or” herein is intended to encompass “and/or” unless otherwise stated.

### Quantitation of Features of Interest in Whole-Slide Imaging

Described herein, in certain embodiments, are computer-implemented methods and systems for eliminating or reducing bias in detection and/or quantification of features of interest present in a plurality of cells in an image. The methods and systems described herein are useful in reducing or eliminating bias in the quantification of features of interest in images by obviating one or more manual procedures, such as finding, labeling, or identifying a cell of interest comprising the feature of interest. In some embodiments, the methods and systems described herein also implement one or more automated methods, which, in some instances, mitigate or reduce human error.

In some embodiments, a computer-implemented method comprises: (a) partitioning, by at least one processor, the image into a plurality of first regions; (b) segmenting, by the at least one processor, each region of the plurality of first regions to identify a first set of features, wherein the segmenting is performed using at least one pixel intensity value relative to a background intensity value; (c) automatically identifying, by the at least one processor, boundaries of the plurality of cells across the plurality of first regions using the at least one pixel intensity value and a pixel location of at least one feature of the first set of features; (d) generating, by the at least one processor, a plurality of second regions using the boundaries of the plurality of cells; (e) segmenting, by the at least one processor, each region of the plurality of second regions to identify the features of interest and quantify or enumerate the features of interest present in a cell of the plurality of cells; and (f) electronically outputting a report indicative of a quantity of the features of interest present in the cell.

Also described herein, in some aspects, are computer-implemented methods and systems for eliminating or reducing bias in the quantification or detection of features of interest, such as nucleic acids (e.g., extrachromosomal DNA (ecDNA), labeled nucleic acids such as fluorescence in situ hybridization (FISH) nucleic acids, homogeneous staining regions (HSR), stained DNA, etc.), that are present in a plurality of cells in a first image. In some embodiments, the method comprises: (a) down-sampling, by at least one processor, the first image, thereby generating a down-sampled image; (b) segmenting, by the at least one processor, the down-sampled image, wherein the segmenting comprises removing, from the down-sampled image, one or more types of nuclei (e.g., compact nuclei, nuclei in interphase of the cell cycle, etc.) originating from the plurality of cells, thereby generating an image devoid of the one or more types of nuclei (e.g., compact nuclei, interphase nuclei); (c) automatically identifying, by the at least one processor, a plurality of first regions in the image devoid of the one or more types of nuclei, wherein each region of the plurality of first regions has a summed pixel intensity value above a threshold intensity value; (d) generating, by the at least one processor, a plurality of contours around at least a subset of the plurality of first regions in the image devoid of the one or more types of nuclei; (e) partitioning, by the at least one processor, the first image into a plurality of second images, using pixel locations of the plurality of contours, wherein each image of the plurality of second images comprises a single region corresponding to a single contour of the plurality of contours of the image devoid of the one or more types of nuclei; and (f) segmenting, by the at least one processor, each image of the plurality of second images to identify one or more nucleic acid features (e.g., ecDNA, FISH probes, HSR) and optionally, quantify or enumerate the one or more nucleic acid features. In some embodiments, the method optionally comprises: (g) electronically outputting a report or information indicative of a quantity or presence of the one or more nucleic acid features present in the plurality of cells in the first image.

Images: In some embodiments, the image (or first image) comprises a plurality of images. In certain embodiments, the plurality of images are images taken from individual regions of a microscope slide, a plate (e.g., cell culture plate), a microwell array, a vial, tube, etc. In some instances, the method further comprises pre-processing operations, such as: overlapping (e.g., using the at least one processor) the plurality of images to generate the image, stitching the plurality of images to generate the image, combining or collating the plurality of images into a sequence of images, etc. In some embodiments, the image comprises a single image of, for example, a region of a microscope slide. In specific embodiments, the image comprises 1, 2, 3, 4, 5, 6, 7, 8, 9, 10, 15, 20, 25, 30, 35, 40, 45, 50, 60, 70, 80, 90, 100, 150, 200, 300, 400, 500, 600, 700, 800, 900, 1000 or more images. In other embodiments, the image comprises at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 150, at least 200, at least 300, at least 400, at least 500, at least 600, at least 700, at least 800, at least 900, at least 1000 or more images. In certain embodiments, the image comprises at most 1000, at most 900, at most 800, at most 700, at most 600, at most 500, at most 400, at most 300, at most 200, at most 100, at most 90, at most 80, at most 70, at most 60, at most 50, at most 45, at most 40, at most 35, at most 30, at most 25, at most 20, at most 15, at most 10, at most 9, at most 8, at most 7, at most 6, at most 5, at most 4, at most 3, at most 2 images or at most 1 image. In some embodiments, the image comprises a numerical range of images, e.g., about 20 to about 100 images.

In some embodiments, the image (or first image) is down-sampled (e.g., using at least one processor) to generate a down-sampled image. In some embodiments, the down-sampling comprises reducing the resolution of the image (or first image), such as by shrinking each dimension of the image (or first image) by a percentage. In some instances, the percentage is between about 70% and about 95%, e.g., about 90%. In some instances, the percentage is about 50%, about 60%, about 70%, about 80%, about 90%, or greater. In some instances, the percentage is at most about 99%, at most about 95%, at most about 90%, at most about 80%, at most about 70%, at most about 60%, or at most about 50%. In some instances, the down-sampling is useful in reducing the memory required of the at least one processor to perform further image processing (e.g., as described herein).

Features of Interest: In some embodiments, the first set of features or the features of interest comprise a macromolecule, e.g., a protein, carbohydrate, lipid, nucleic acid molecule, or a combination thereof. In some instances, the first set of features or the features of interest comprise a nucleic acid molecule, which is optionally labeled. In certain embodiments, the first set of features or the features of interest comprise a deoxyribonucleic acid (DNA) molecule. In some instances, the first set of features or the features of interest comprise extrachromosomal DNA (ecDNA). In some instances, the first set of features or the features of interest comprises circular ecDNA. In some instances, the first set of features or the features of interest comprise chromosomal DNA. In some instances, the first set of features or the features of interest comprise probes. In some embodiments, the probes are gene-specific probes, and are optionally labeled (e.g., fluorescently or colorimetrically). In certain embodiments, the gene-specific probes comprise labeled probes used, for instance, in in situ hybridization assays. In some embodiments, the gene-specific probes are located on or label a portion of ecDNA. One or more gene-specific probes can be used—for example, the ecDNA is labeled with at least two gene-specific probes, each of which hybridize to a different feature (e.g., gene-specific sequence). In some instances, the probes have different colorimetric or fluorescence (e.g., different wavelengths of excitation and/or emission). Accordingly, in some instances, each probe is quantified, detected, or identified. In some embodiments, the gene-specific probe comprises fluorescently labeled gene-specific probes for use in a fluorescence in situ hybridization (FISH) assay. In another embodiment, the gene-specific probe comprises colorimetrically labeled gene-specific probes for use in a colorimetric in situ hybridization (CISH) assay. In some instances, the features of interest comprise a chromosomal and/or ecDNA homogeneous staining region (HSR). In some instances, the features of interest comprise one or more labeled nucleic acid molecules (e.g., from a FISH or CISH assay, HSRs). In some instances, the features of interest comprise one or more nucleic acid molecules co-labeled with two more probes (e.g., from a FISH or CISH assay), for example to detect two or more gene amplifications, genes, or loci associated with ecDNA. In some instances, the features of interest comprise one or more nucleic acid molecules arising from one or more gene amplifications. In some instances, the features of interest comprise a cell nucleus in metaphase or a spread of metaphase chromosomes (also referred herein as “metaphase spread”).

The in situ hybridization assay may be used for a variety of purposes. In some instances, the in situ hybridization assay is used to establish the presence of a nucleic acid sequence (e.g., a gene) in a cell or plurality of cells. In some instances, the in situ hybridization assay is used to establish the presence of a nucleic acid sequence (e.g., a gene) in a feature of interest, e.g., whether a gene is present on a non-chromosomal DNA molecule (e.g., ecDNA). In such an example, the in situ hybridization assay is used in addition to the quantitation of the features of interest to yield information on, for instance, the number or distribution of features of interest (e.g., ecDNA) that comprise a particular gene, the ratio of the presence of the gene on a subset of features of interest compared to a different subset of features of interest (e.g., the ratio of the gene on ecDNA compared to chromosomal DNA), the co-localization of features of interest (e.g., co-localization of two different gene amplifications on a chromosome or on ecDNA), etc.

In some embodiments, the labeled probes comprise protein-specific, lipid-specific, or carbohydrate-specific probes, which are optionally labeled (e.g., fluorescently or colorimetrically). In some embodiments, the probe comprises an antibody, antibody fragment, affimer, aptamer, binding protein, antibody-mimetic protein (e.g., designed ankyrin repeat protein), lipid-binding agent, etc. In some embodiments, the features of interest described herein include one or more types of probes (e.g., labeled probes).

In some instances, the image comprises one or more cells that do not comprise the features of interest. In some embodiments, the features of interest are ecDNA, and the image comprises one or more cells that do not comprise ecDNA and thus are devoid of the features of interest. In some instances, the image comprises a mixture of cells, some of which comprise the features of interest and some of which do not comprise the features of interest. In some instances, the image comprises a cell having multiple types of features of interest. For example, the features of interest may include a labeled probe (e.g., FISH or CISH probe, labeled antibody), or more than one labeled probe, and/or may include features on both ecDNA and chromosomal DNA.

Cells: The cell or plurality of cells may comprise any cell type of interest. In some instances, the cell is from a cell line, cell culture, or a primary source (e.g., a tumor or tissue sample). Non-limiting examples of cells include prokaryotic cells, eukaryotic cells, bacterial, fungal, plant, mammalian, or other animal cell types, mycoplasmas, normal tissue cells, tumor cells, or any other cell type, whether derived from single-cell or multicellular organisms. In some embodiments, the cell or plurality of cells is mammalian. In some embodiments, the cell or plurality of cells is from a tumor. In some instances, the cell is alive. In some instances, the cell is dead. In some instances, the cell is fixed, e.g., using a fixative such as methanol, formaldehyde, or paraformaldehyde. In some instances, the cell is permeabilized.

In some instances, the cell or plurality of cells comprises a mixture of cells that are in varying stages of the cell cycle. In some embodiments, a cell or a plurality of cells in the mixture comprises cells that are in interphase or undergoing cell division. In some instances, the cell or plurality of cells comprise any cell that is in any stage of cell division, e.g., prophase, prometaphase, metaphase, anaphase, telophase, or cytokinesis. In certain embodiments, the cells comprising the features of interest are cells in interphase or metaphase. In certain embodiments, the cells comprising the feature of interest are metaphase cells, and one or more processes describe herein are used to remove cells (or nuclei) that are not in metaphase.

Statistical Operations: In some instances, the method further comprises performing a statistical operation. In some instances, the statistical operation is performed on the first set of features or the features of interest. In some instances, the statistical operation is performed at any useful step of the method, e.g., prior to, during, or following segmentation, prior to, during, or following identification of the boundaries of the cells, prior to, during, or following generation of the second regions using the boundaries of the cell, prior to, during, or following segmentation to identify and quantify, enumerate, or label the features of interest, prior to, during, or following output of the report, etc. In some instances, more than one statistical operation is performed (e.g., during different processes).

In some embodiments, a statistical operation is performed to automatically identify the boundaries of the cell or plurality of cells across the plurality of regions (e.g., first regions), or to identify overlapping regions with a summed pixel intensity above a threshold and to generate a contour comprising or surrounding the overlapping regions. In some embodiments, the statistical operation comprises using or applying a statistical clustering algorithm of the at least one pixel intensity value (e.g., during image segmentation or boundary identification) or pixel locations or coordinates to identify the boundaries of the cells or contours around metaphase spreads, e.g., using the overlapping regions (or overlapping windows). In some embodiments, the statistical clustering algorithm is any useful statistical clustering algorithm, such as Gaussian mixture models, k-means clustering, density-based spatial clustering of applications with noise (DBSCAN), ordering points to identify the clustering structure (OPTICS), etc. In some instances, the boundaries or contours of the cells across the plurality of regions are labeled or identified based on the proximity or clustering of the identified features (e.g., the first set of features, the overlapping regions having a summed pixel intensity above the threshold value).

In some instances, the statistical operation comprises combining an overlapping clustering of the cell boundaries (e.g., those identified in (c) using the at least one pixel intensity value) to generate a plurality of second regions, where each of the plurality of second regions contains a single cell. Such an overlapping process is useful in identifying, for instance, a cell that spans across multiple images or first regions. In such examples, the pixel location or coordinates of the first set of features are used to identify the boundaries of the plurality of cells, and subsequently, the pixel location or coordinates of the boundaries are used to determine if a cell spans across one or more regions of the first regions. A plurality of second regions are then generated using the boundaries (and/or coordinates or locations thereof), and each region of the plurality of second regions comprises a single cell.

In a non-limiting example, a cell comprising the features of interest spans across multiple regions of the set of first regions. Following segmentation of each region of the first regions, the one or more processors map or contain information relating to the pixel locations or coordinates of each of the identified first set of features. As such, the pixel locations or coordinates of the first set of features of one region of the first regions are overlapped with the pixel locations or coordinates of the first set of features identified in another region of the first regions. The pixel locations or coordinates of each region of the first regions, or the distributions of the pixel locations or coordinates, are then used to identify the boundaries of cell spanning the multiple regions. Thereafter, a second region is generated containing all the overlapping first set of features or identified boundaries, e.g., a second region that comprises the entire cell.

In some instances, a statistical operation is performed on the features of interest that are identified during image segmentation (e.g., image segmentation of the second regions to identify the features of interest in each region of the second regions). In certain instances, the statistical operation compares a pixel intensity and pixel location of a first subset of the features of interest to a pixel intensity and pixel location of a second subset of the features of interest. Based on the comparison, the statistical operation is used to remove outliers or potentially false-positive signals (e.g., debris, dust, or noise). In some instances, the features of interest identified during image segmentation comprise a first subset of features that comprise ecDNA (the “true” feature of interest) and also debris (a “false” feature of interest), while a second subset of features comprise chromosomal DNA. In such an example, the statistical operation compares the pixel intensity and/or location of the first subset of features (containing ecDNA and debris) to the pixel intensity and/or location of the second subset of features (containing chromosomal DNA). Based on the comparison, the statistical operation determines the distribution of the pixel locations to determine overlap. Thereafter, the overlap is used to determine whether the locations of the first subset of features sufficiently overlap with the second subset of features. The features from the first subset of features that sufficiently overlap with the second subset of features are marked as “true” features of interest (e.g., ecDNA), whereas those that do not sufficiently overlap are marked as “false” features of interest (e.g., debris) and are removed as outliers.

Image segmentation: In some instances, image segmentation is used to identify or classify features or objects (e.g., a first set of features or features of interest). In some embodiments, the image segmentation process comprises measuring or obtaining a pixel intensity value of each pixel in an image and comparing the pixel intensity value of at least a subset of pixels to a reference pixel value. In some instances, the reference pixel value is a background pixel value. In some instances, the reference pixel value is a pixel value from a different image. In some instances, the pixel intensity values of an image are background subtracted (e.g., subtracting a background pixel intensity value from the intensity values of the subset of pixels) or normalized to a reference pixel value (e.g., background pixel intensity). In some instances, the image segmentation comprises a classification procedure. For example, the image segmentation may apply a threshold (e.g., to the background-subtracted pixel intensity values) to generate a binary mask, which identifies or classifies each pixel, or a cluster of pixels, as having a pixel intensity value above or below the threshold. Pixels that have intensity values above the threshold value are marked as potentially being a higher-than-background intensity region, and such identification or classification are used for identification or classification of the first set of features or the features of interest.

In some instances, the image segmentation comprises alternative or additional processes or operations. For instance, the image or product of image segmentation (e.g., binary mask) is subjected to further image processing, which includes, for instance, transformations (e.g., watershed processing), edge detection (e.g., Canny edge detection), blurring or deblurring, texturing, clustering, etc. It will be appreciated that multiple image processing algorithms may be implemented (e.g., by the one or more processors) for refinement of the identification and quantitation of the features of interest.

In some instances, the image segmentation comprises a filtering operation, e.g., for removal of particular features from the image or down-sampled image. In some examples, the filtering operation comprises white top -hat filtering, which, in some instances, includes performing (e.g., using at least one processor) a morphological opening. The morphological opening performs an erosion, followed by dilation. Subsequently, the morphological opening is subtracted from the down-sampled image (e.g., by removing pixels belonging to the morphological opening). In some instances, the white top-hat filtering is used to remove particular features of a smaller size or features with high pixel intensity, such as noise, punctae, debris, or other features. For example, the small features with high pixel intensity may be compared, using the processor, to a threshold intensity value, and pixels having a pixel intensity above the threshold intensity are removed. Similarly, a size filter can be applied, such that a cluster of pixels below a size threshold are removed. In some instances, the white top-hat filtering comprises multiple operations and is used to first remove chromosomes or ecDNA, identify larger features with a high pixel intensity (e.g., intact nuclei, interphase nuclei, compact nuclei), and then remove the identified larger features with a high pixel intensity (e.g., compact nuclei) from the down-sampled image. Accordingly, the white top-hat filter is useful in removing noise and compact nuclei or other non-metaphase cells. In some instances, the resultant image from the white top-hat filtering comprises cells in metaphase, with the compact nuclei removed (e.g., a compact-nuclei-free image).

In some instances, an additional filtration operation is performed on the compact-nuclei-free image, e.g., for classification, clustering, or identification of metaphase spreads from individual cells. In some instances, the additional filtration operation outputs a plurality of first regions, in which each region of the plurality of first regions has a summed pixel intensity value above a threshold intensity value. In some instances, the additional filtration operation is used to identify regions of high pixel intensity (e.g., relative to background), in the compact-nuclei-free image. In some instances, the additional filtration operation comprises producing a window and sliding the window across the compact-nuclei-free image. As the window slides across the image (e.g., at each pixel location of the image), a summation of pixel intensities in the window is performed. In instances in which the summation of pixel intensities is higher than the threshold value, the window is marked or identified, using the processor, as region of interest (e.g., a first region of a plurality of regions). Such a region of interest is potentially indicative of a portion of a metaphase cell. Subsequently, the overlapping regions of interest are grouped together. In some instances, a contour is generated around a subset of the identified regions of interest. For example, first regions (regions of interest) that overlap in pixel location or coordinates are grouped together and subjected to contouring, which generates a contour or boundary surrounding or encompassing the overlapping regions. In some instances, a statistical operation, such as those described above, is performed to generate the contours. In some instances, one or more pixel locations or coordinates of the generated contours (e.g., edges or boundaries, centroids or center of mass) are output and subsequently used for further image processing. Each contour may represent a single cell, a single metaphase spread, or overlapping features or regions of interest.

The window size may be any appropriate or useful size for identifying the first regions (regions of interest). For example, in some instances, the window kernel size is 16 pixels by 16 pixels. In some instances, the window kernel shape is non-square (e.g., rectangular, rhomboidal, circular, triangular, etc.). In some instances, the window kernel size is variable or adjustable. In some instances, the window kernel size is larger (e.g., 20 pixels x 20 pixels, 30 pixels x 30 pixels, etc.) or smaller (e.g., 10 pixels x 10 pixels). A range of window kernel sizes is possible, e.g., 16 pixels x 20 pixels, 32 pixels x 32 pixels, etc. In some instances, the output pixel locations or coordinates of the generated contours are used (e.g., by the one or more processors) to further process the original input image (e.g., the first image), such as to identify features of interest (e.g., ecDNA, FISH probes, HSR) in the original image. For example, in some instances, the pixel locations or coordinates of each contour of the plurality of contours is mapped to the original image to generate a plurality of second images (or regions of the first image), each of which comprises a region corresponding to a single contour of the compact-nuclei-free image. In one such example, the at least one processor obtains the pixel locations or coordinates of each contour from the compact-nuclei-free image and uses them to isolate or partition a region of the original image that corresponds to the same pixel location or coordinates of the compact-nuclei-free image. In instances in which a downsampling has been performed, the mapping of the coordinates or pixel locations from the compact-nuclei-free image (down-sampled) to the original image (not down-sampled) may be accounted for (e.g., by performing a transformation or scaling by the down-sampling factor). The resultant plurality of second images each comprise a metaphase spread, which in some instances, is located in the center of the image. In some instances, further segmentation is performed, to identify and/or quantify (or label or enumerate) the features of interest (e.g., ecDNA, FISH probes).

In some instances, one or more processes disclosed herein (e.g., image segmentation and/or identification of features) involves using or applying a deep learning algorithm to classify features. For example, in certain instances, image segmentation is used to identify regions of high pixel intensity value (e.g.., compared to a background value), and the deep learning algorithm, which is part of the image segmentation process or is a separate process, is used to cluster or classify the regions into one or more classifications. The classifications are based, for example, on a property of the regions with high pixel intensity value. In some instances, the classifications are based on the proximity of the high-intensity pixels to other high-intensity pixels, shapes, contours, etc. of each region. In some instances, the deep learning algorithm comprises functions to smooth, blur, or overlap pixels or group them (e.g., into a cluster of high-intensity pixels). In some instances, the deep learning algorithm is trained using expert-identified features of interest (e.g., known or expert-identified images of ecDNA). In some instances, the deep learning algorithm outputs or is trained to output confidence (e.g., a confidence interval) in each identified feature. In some instances, the confidence can be used as a flag or demarcation to indicate a feature that requires additional review or selection (e.g., by a user). In some instances, one or more classifications arising from the deep learning algorithm is then used for the statistical operation, to identify, for instance, the “true” features of interest (e.g., ecDNA) from the “false” features of interest (e.g., debris, noise), as described herein.

Output: In some instances, the computer-implemented methods and systems disclosed herein output information on the plurality of cells in the input image. In some instances, the output information indicates the presence of one or more nucleic acid features or features of interest (e.g., metaphase nuclei, ecDNA, HSR, FISH probes, or other labeled nucleic acids). In some instances, the output information indicates a quantity of the one or more features of interest present in the plurality of cells or a number of cells (or percentage of cells) that have the feature of interest. In some instances, the output information is indicative of ratios of features of interest. For example, the output information may comprise a quantity of FISH probes on an HSR relative to a quantity of FISH probes on the ecDNA. In another example, the output information may comprise a quantity of HSR on a native chromosome and a quantity of HSR on ecDNA. In yet another example, the output information may comprise a ratio of pixel intensity of HSR on chromosomes relative to pixel intensity of FISH probes on ecDNA or chromosomal DNA. In instances where ecDNA is detected, the ecDNA is quantitated, and other properties of the ecDNA are assessed, such information may be optionally outputted as an electronic output, e.g., in a report. In certain examples, the report comprises other signatures or statistics of the image (e.g., average number of ecDNA per cell, spatial locations of ecDNA relative to chromosomal DNA, location of outliers, etc.). In some instances, the output is a report, which can comprise a text file, a graph or plot, a comma-separated values (csv) file, or other report.

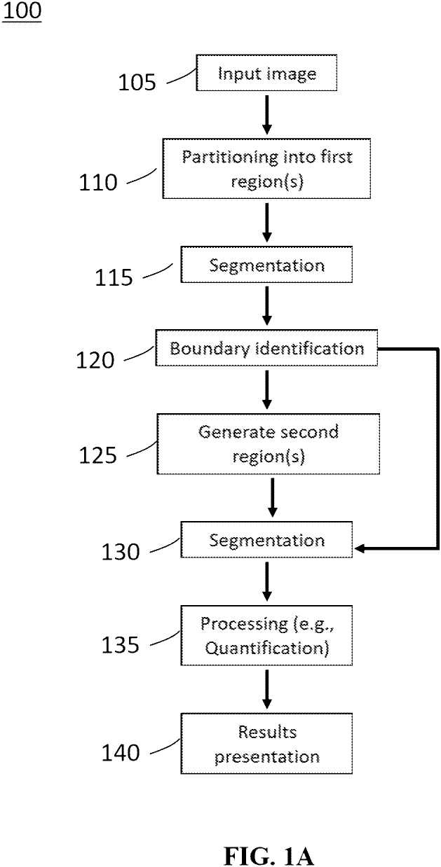

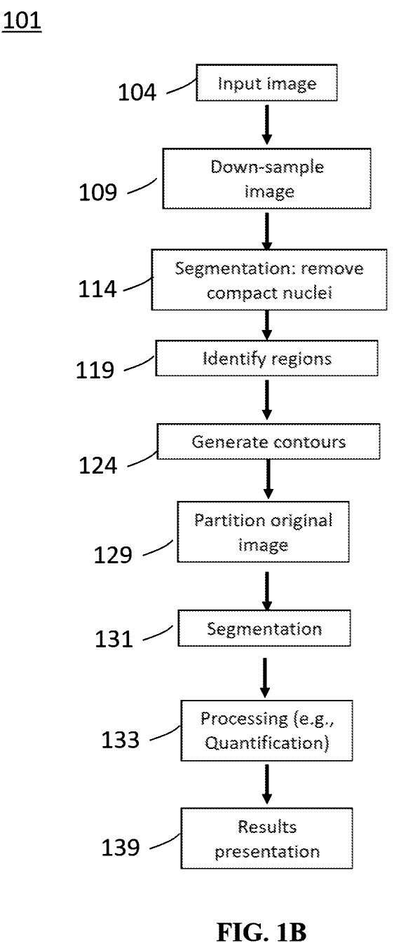

FIG. 1A shows an example workflow 100 of several of the methods and processes described herein. The workflow 100 uses an input image 105, which in certain examples, includes an image of an entire or a portion of a microscope slide containing cells having a feature of interest (e.g., ecDNA), an image comprising a plurality of overlapped or stitched images of a microscope slide containing cells having a feature of interest (e.g., ecDNA), etc. In process 110, the input image 105 is partitioned, e.g., using one or more processors, into a plurality of first regions. Each region of these first regions is subjected to process 115, which includes image segmentation. During image segmentation, a first set of features (e.g., ecDNA, ecDNA-like structures, etc.) are identified or labeled based on the pixel intensity value relative to a background intensity value. In some instances, the image segmentation comprises using a deep learning algorithm to identify or label the features of interest (e.g., ecDNA). In some instances, the deep learning algorithm is trained using, for instance, expert-identified features of interest (e.g., ecDNA). Following segmentation, in process 120, the boundaries of the cells are identified or labeled using the one or more processors. The boundaries are identified or labeled using a pixel intensity value or a plurality of pixel intensity values and a pixel location or a plurality of pixel locations of at least one feature of the first set of features (e.g., ecDNA, ecDNA-like structures, etc.). In process 125, the boundaries identified or labeled in process 120 are used to generate second regions. For instance, if a cell spans across multiple regions of the first set of regions, following segmentation and boundary identification or labeling, the one or more processors clusters the overlapping regions based on the pixel locations of the boundaries of each of the first regions, thereby determining that the cell spanned across multiple of the first regions. The processor then generates a second region that comprises the entire cell. In such an example, each second region comprises a single cell. In process 130, the second regions are subjected to another image segmentation. In some instances, the image segmentation of process 130 is substantially similar to that in process 115 and is used to identify or label the features of interest in the second regions, each of which comprises a single cell. In process 135, further processing is implemented, e.g., using at least one processor. The further processing includes, for instance, quantification of the features of interest. In some instances, the further processing comprises using a statistical operation. In some instances, the statistical operation compares a pixel intensity or location of a subset of a subset of the features of interest to a pixel intensity or location of an additional subset of the features of interest. In some instances, the statistical operation compares a distribution of pixel intensities or locations of a subset of a subset of the features of interest to a distribution of pixel intensities or locations of an additional subset of the features of interest. Using such a comparison, or by determining sufficient colocalization of the features of interest (e.g., proximity of the ecDNA and ecDNA-like structures to the chromosomal DNA), the statistical operation is used to remove outliers (e.g., dust, debris, or other noise). In process 140, the results of process 135 are output or displayed. In some instances, the results are output or displayed via a graphical user interface (GUI). In some instances, the results are output as numerical values, a text-based report comprising the number of features of interest, and/or other information (e.g., pixel locations, pixel intensities, size of features, shape of features, statistics on the features of interest, etc.).

In some instances, the methods and processes described herein require a fewer or a greater number of operations. In certain instances, referring to FIG. 1A, the workflow 100 comprises processes 105, 110, 115, 120, 130, 135, and 140. In such an example, an input image is partitioned into first regions and the first regions are segmented to identify a first set of features, which are subsequently used for boundary identification. The identified boundaries of the cells are then re-segmented and processed to identify and quantify and/or enumerate (or label) the features of interest, prior to results presentation. In another example, the workflow 100 comprises processes 105, 110, 115, 135, and 140. In such examples, an input image is partitioned into first regions and the first regions are segmented to identify the features of interest without intermediary operations. The identified features of interest are then processed (e.g., quantified) and the results are presented (e.g., electronically). It will be appreciated that, while various operations are disclosed herein (e.g., in FIG. 1A), the methods and processes disclosed herein may include some, all, or additional operations.

FIG. 1B shows an example workflow 101 of several of the methods and processes described herein. The workflow 101 uses an input image 104, which in certain examples, includes an image of an entire or a portion of a microscope slide containing cells having a feature of interest (e.g., ecDNA), an image comprising a plurality of overlapped or stitched images of a microscope slide containing cells having a feature of interest (e.g., ecDNA), etc. In process 109, the input image is down-sampled, e.g., using one or more processors, into a down-sampled image with reduced resolution. The down-sampled image is subjected to process 114, which includes image segmentation. During image segmentation, one or more compact nuclei originating from the cells are removed from the down-sampled image (e.g., using white top-hat filtering or other segmentation or filtration processes), thus generating a compact-nuclei-free image. In some instances, process 114 is also used to remove high-intensity punctae, noise, debris, etc. Following segmentation, in process 119, a plurality of first regions is identified or labeled using the one or more processors. Each region of the plurality of first regions has a summed pixel intensity value that is above a threshold intensity value. In some instances, the plurality of first regions is identified by using a sliding window (e.g., a 16 pixel x 16 pixel window) across the compact-nuclei-free image, summing the pixel intensity values in the window at each location across the image, and then thresholding the locations in which the summed pixel intensity value is above the threshold value. In some instances, the one or more processors labels or marks the windows that have a summed pixel intensity value above the threshold (e.g., the processor marks the windows above the threshold intensity value as a region of interest). In process 124, contours surrounding or comprising the overlapping regions of the plurality of first regions are generated. The locations or coordinates (e.g., centroid) of each contour is also generated. In process 129, the coordinates or locations of each contour are used to partition a plurality of second images (or regions) from the original, input image. In some instances, each of the second images comprises a region corresponding to a single contour (e.g., corresponding to a metaphase spread or a single cell). In other instances, the second images comprise more than one contour. In process 131 the second images are subjected to another image segmentation. In some instances, the image segmentation of process 131 is substantially similar to that in process 114. In other instances, the image segmentation of process 131 is different and is used to identify or label the features of interest (e.g., ecDNA, FISH probes, etc.) in the second images, each of which comprises a single cell or single metaphase spread. In some instances, the image segmentation of process 131 comprises using a deep learning algorithm to identify or label the features of interest (e.g., ecDNA). In some instances, the deep learning algorithm is trained using, for instance, expert-identified features of interest (e.g., ecDNA).

In process 133, further processing is implemented, e.g., using at least one processor. The further processing includes, for instance, quantification of the features of interest (e.g., ecDNA, FISH probes, etc.). In some instances, the further processing comprises using a statistical operation. In some instances, the statistical operation compares a pixel intensity or location of a subset of a subset of the features of interest to a pixel intensity or location of an additional subset of the features of interest. In some instances, the statistical operation compares a distribution of pixel intensities or locations of a subset of a subset of the features of interest to a distribution of pixel intensities or locations of an additional subset of the features of interest. Using such a comparison, or by determining sufficient colocalization of the features of interest (e.g., proximity of the ecDNA and ecDNA-like structures to the chromosomal DNA), the statistical operation is used to remove outliers (e.g., dust, debris, or other noise). In process 139, the results of process 133 are output or displayed. In some instances, the results are output or displayed via a graphical user interface (GUI). In some instances, the results are output as numerical values, a text-based report comprising the number of features of interest, and/or other information (e.g., pixel locations, pixel intensities, size of features, shape of features, statistics on the features of interest, etc.).

Systems: In another aspect, provided herein is a computer-implemented system for performing unbiased, automatic quantification of features of interest present in a plurality of cells in an image, comprising: at least one processor configured to perform executable instructions and a memory comprising the executable instructions, which, when executed by the at least one processor, causes the at least one processor to: (a) partition the image into a plurality of first regions; (b) segment each of the plurality of first regions to identify a first set of features, wherein the segmenting is performed using at least one pixel intensity value relative to a background intensity value; (c) identify boundaries of the plurality of cells using the at least one pixel intensity value and a pixel location of at least one feature of the first set of features; (d) generate a plurality of second regions using the boundaries of the plurality of cells; (e) segment each of the plurality of second regions to identify the features of interest and quantify or enumerate the features of interest present in a cell of the plurality of cells; and (f) electronically output a report indicative of a quantity of the features of interest present in the cell.

In another aspect, disclosed herein is a computer-implemented system for performing non-biased, automatic detection of nucleic acids present in a plurality of cells in a first image, comprising: at least one processor configured to perform executable instructions and a memory comprising the executable instructions, which, when executed by the at least one processor, causes the at least one processor to: (a) down-sample the first image, thereby generating a down-sampled image; (b) segment the down-sampled image, wherein the segmenting comprises removing, from the down-sampled image, one or more compact nuclei originating from the plurality of cells, thereby generating a compact-nuclei-free image; (c) automatically identify a plurality of first regions in the compact-nuclei-free image, wherein each region of the plurality of first regions has a summed pixel intensity value above a threshold intensity value; (d) generate a plurality of contours around at least a subset of the plurality of first regions in the compact-nuclei-free image; (e) partition the first image into a plurality of second images, using pixel locations of the plurality of contours, wherein each image of the plurality of second images comprises a single region corresponding to a single contour of the plurality of contours of the compact-nuclei-free image; (f) segment each image of the plurality of second images to identify one or more nucleic acid features; and (g) electronically output information indicative of the presence or quantity of the one or more nucleic acid features present in the plurality of cells in the first image.

Non-transitory computer readable storage media: In another aspect, disclosed herein is a non-transitory computer readable storage medium encoded with a computer program including instructions executable by a processor to perform non-biased, automatic quantification of features of interest present in a plurality of cells in an image, the computer program comprising: (a) a software module for partitioning the image into a plurality of first regions; (b) a software module for segmenting each of the plurality of first regions to identify a first set of features, wherein the segmenting is performed using at least one pixel intensity value relative to a background intensity value; (c) a software module for automatically identifying boundaries of the plurality of cells across the plurality of first regions using the at least one pixel intensity value and a pixel location of at least one feature of the first set of features; (d) a software module for generating a plurality of second regions using the boundaries of the plurality of cells; (e) a software module for segmenting each of the plurality of second regions to identify the features of interest and quantify (or enumerate) the features of interest present in a cell of the plurality of cells; and (f) a software module for electronically outputting a report indicative of a quantity of the features of interest present in the cell.

In another aspect, provided herein is a non-transitory computer readable storage medium encoded with a computer program including instructions executable by a processor to perform non-biased, automatic detection of nucleic acids present in a plurality of cells in a first image, the computer program comprising: (a) a software module for down-sampling the first image, thereby generating a down-sampled image; (b) a software module for segmenting the down-sampled image, wherein the segmenting comprises removing, from the down-sampled image, one or more compact nuclei originating from the plurality of cells, thereby generating an compact-nuclei-free image; (c) a software module for automatically identifying a plurality of first regions in the compact-nuclei-free image, wherein each region of the plurality of first regions has a summed pixel intensity value above a threshold intensity value; (d) a software module for generating a plurality of contours around at least a subset of the plurality of first regions in the compact-nuclei-free image; (e) a software module for partitioning the first image into a plurality of second images, using pixel locations of the plurality of contours, wherein each image of the plurality of second images comprises a single region corresponding to a single contour of the plurality of contours of the compact-nuclei-free image; (f) a software module for segmenting each image of the plurality of second images to identify one or more nucleic acid features; and (g) a software module for electronically outputting information indicative of the presence or quantity of the one or more nucleic acid features present in the plurality of cells in the first image.

Alternative computer-implemented methods: In another aspect of the present disclosure, provided herein is a computer-implemented method of eliminating bias in a quantification of features of interest present in a plurality of cells in an image, the computer-implemented method comprising: (a) partitioning, by at least one processor, the image into a plurality of first regions; (b) segmenting, by the at least one processor, each of the plurality of first regions to identify a first set of features, wherein the segmenting is performed using at least one pixel intensity value relative to a background intensity value; (c) automatically identifying, by the at least one processor, boundaries of the plurality of cells using the at least one pixel intensity value and a pixel location of at least one feature of the first set of features across the plurality of first regions; (d) segmenting, by the at least one processor, a plurality of second regions defined by the boundaries to identify the features of interest and quantify or enumerate the features of interest present in a cell of the plurality of cells; and (e) electronically outputting a report indicative of a quantity of the features of interest present in the cell.

### Computing System

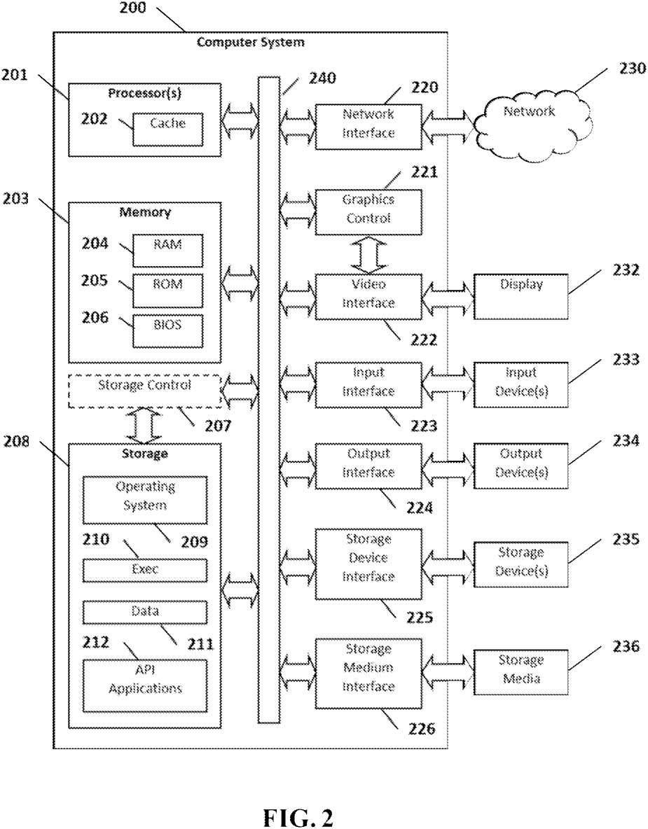

Referring to FIG. 2, a block diagram is shown depicting an exemplary machine that includes a computer system 200 (e.g., a processing or computing system) within which a set of instructions causes a device to perform or execute any one or more of the aspects and/or methodologies of the present disclosure. The components in FIG. 2 are examples only and do not limit the scope of use or functionality of any hardware, software, embedded logic component, or a combination of two or more such components implementing particular embodiments.

Computer system 200 may include one or more processors 201, a memory 203, and a storage 208 that communicate with each other, and with other components, via a bus 240. The bus 240 may also link a display 232, one or more input devices 233 (which may, for example, include a keypad, a keyboard, a mouse, a stylus, etc.), one or more output devices 234, one or more storage devices 235, and various tangible storage media 236. All of these elements may interface directly or via one or more interfaces or adaptors to the bus 240. In some instances, the various tangible storage media 236 interfaces with the bus 240 via storage medium interface 226. Computer system 200 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 200 includes the one or more processor(s) 201 (e.g., central processing units (CPUs), general-purpose graphics processing units (GPGPUs), or quantum processing units (QPUs)) that carry out functions. Processor(s) 201 optionally contains a cache memory unit 202 for temporary local storage of instructions, data, or computer addresses. Processor(s) 201 are configured to assist in the execution of computer-readable instructions. Computer system 200 may provide functionality for the components depicted in FIG. 2 as a result of the processor(s) 201 executing non-transitory, processor-executable instructions embodied in one or more tangible computer-readable storage media, such as memory 203, storage 208, storage devices 235, and/or storage medium 236. The computer-readable media may store software that implements particular embodiments, and processor(s) 201 may execute the software. Memory 203 may read the software from one or more other computer-readable media (such as mass storage device(s) 235, 236) or from one or more other sources through a suitable interface, such as network interface 220. The software may cause processor(s) 201 to carry out one or more processes or one or more steps of one or more processes described or illustrated herein. Carrying out such processes or steps may include defining data structures stored in memory 203 and modifying the data structures as directed by the software.

The memory 203 may include various components (e.g., machine-readable media) including, but not limited to, a random access memory component (e.g., RAM 204) (e.g., static RAM (SRAM), dynamic RAM (DRAM), ferroelectric random access memory (FRAM), phase-change random access memory (PRAM), etc.), a read-only memory component (e.g., ROM 205), and any combinations thereof. ROM 205 may act to communicate data and instructions unidirectionally to processor(s) 201, and RAM 204 may act to communicate data and instructions bidirectionally with processor(s) 201. ROM 205 and RAM 204 may include any suitable tangible computer-readable media described below. In one example, a basic input/output system 206 (BIOS), including basic routines that help to transfer information between elements within computer system 200, such as during start-up, may be stored in the memory 203.

Fixed storage 208 is connected bidirectionally to processor(s) 201, optionally through storage control unit 207. Fixed storage 208 provides additional data storage capacity and may also include any suitable tangible computer-readable media described herein. Storage 208 may be used to store operating system 209, executable(s) 210, data 211, applications 212 (application programs), and the like. Storage 208 also includes, for instance, an optical disk drive, a solid-state memory device (e.g., flash-based systems), or a combination of any of the above. Information in storage 208 may, in appropriate instances, be incorporated as virtual memory in memory 203.

In one example, storage device(s) 235 may be removably interfaced with computer system 200 (e.g., via an external port connector (not shown)) via a storage device interface 225. Particularly, storage device(s) 235 and an associated machine-readable medium may provide nonvolatile and/or volatile storage of machine-readable instructions, data structures, program modules, and/or other data for the computer system 200. In one example, software may reside, completely or partially, within a machine-readable medium on storage device(s) 235. In another example, software may reside, completely or partially, within processor(s) 201.

Bus 240 connects a wide variety of subsystems. Herein, reference to a bus may encompass one or more digital signal lines serving a common function, where appropriate. Bus 240 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 200 may also include an input device 233. In one example, a user of computer system 200 may enter commands and/or other information into computer system 200 via input device(s) 233. Examples of an input device(s) 233 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 233 may be interfaced to bus 240 via any of a variety of input interfaces 223 (e.g., input interface 223) including, but not limited to, serial, parallel, game port, USB, FIREWIRE, THUNDERBOLT, or any combination of the above.

In particular embodiments, when computer system 200 is connected to network 230, computer system 200 may communicate with other devices, specifically mobile devices and enterprise systems, distributed computing systems, cloud storage systems, cloud computing systems, and the like, connected to network 230. Communications to and from computer system 200 may be sent through network interface 220. For example, network interface 220 may receive incoming communications (such as requests or responses from other devices) in the form of one or more packets (such as Internet Protocol (IP) packets) from network 230, and computer system 200 may store the incoming communications in memory 203 for processing. Computer system 200 may similarly store outgoing communications (such as requests or responses to other devices) in the form of one or more packets in memory 203 and communicated to network 230 from network interface 220. Processor(s) 201 may access these communication packets stored in memory 203 for processing.

Examples of the network interface 220 include, but are not limited to, a network interface card, a modem, and any combination thereof. Examples of a network 230 or network segment 230 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 230, may employ a wired and/or a wireless mode of communication. In general, any network topology may be used.

In some instances, information and data are displayed through a display 232. Examples of a display 232 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 232 may interface to the processor(s) 201, memory 203, and fixed storage 208, as well as other devices, such as input device(s) 233, via the bus 240. The display 232 is linked to the bus 240 via a video interface 222, and transport of data between the display 232 and the bus 240 may be controlled via the graphics control 221. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 232, computer system 200 may include one or more other peripheral output devices 234 including, but not limited to, an audio speaker, a printer, a storage device, and any combinations thereof. Such peripheral output devices may be connected to the bus 240 via an output interface 224. Examples of an output interface 224 include, but are not limited to, a serial port, a parallel connection, a USB port, a FIREWIRE port, a THUNDERBOLT port, and any combinations thereof.

In addition, or as an alternative, computer system 200 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general-purpose processor, a digital signal processor (DSP), an application-specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by the one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor reads information from, and writes information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device’s hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony®, PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer-readable storage medium is a tangible component of a computing device. In still further embodiments, a computer-readable storage medium is optionally removable from a computing device. In some embodiments, a computer-readable storage medium includes, by way of non-limiting examples, CD-ROMs, DVDs, flash memory devices, solid-state memory, magnetic disk drives, magnetic tape drives, optical disk drives, distributed computing systems including cloud computing systems and services, and the like. In some instances, the program and instructions are permanently, substantially permanently, semi-permanently, or non-transitorily encoded on the media.

### Computer Program

In some embodiments, the platforms, systems, media, and methods disclosed herein include at least one computer program or use of the same. A computer program includes a sequence of instructions, executable by one or more processor(s) of the computing device’s CPU, written to perform a specified task. Computer-readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer-readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object-oriented, associative, XML, and document-oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

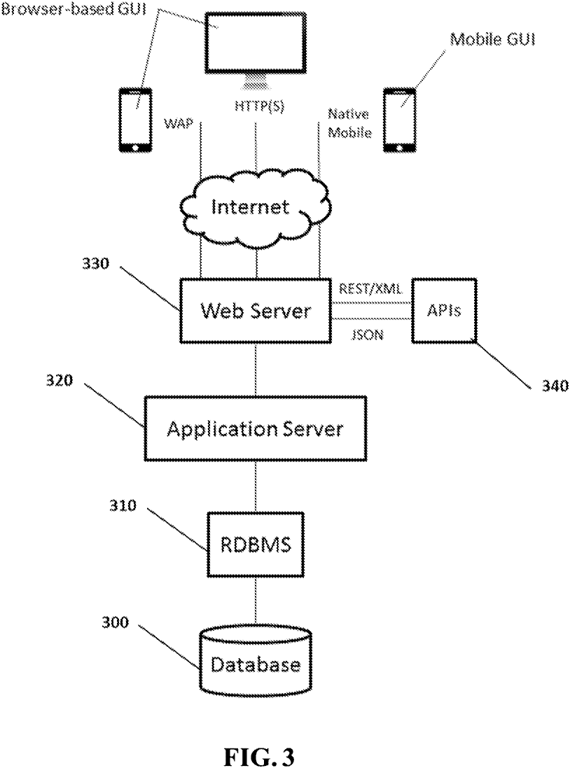

Referring to FIG. 3, in a particular embodiment, an application provision system comprises one or more databases 300 accessed by a relational database management system (RDBMS) 310. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application servers 320 (such as Java servers, .NET servers, PHP servers, and the like) and one or more web servers 330 (such as Apache, IIS, GWS and the like). The web server(s) optionally expose(s) one or more web services via app application programming interfaces (APIs) 340. Via a network, such as the Internet, the system provides browser-based and/or mobile native user interfaces.

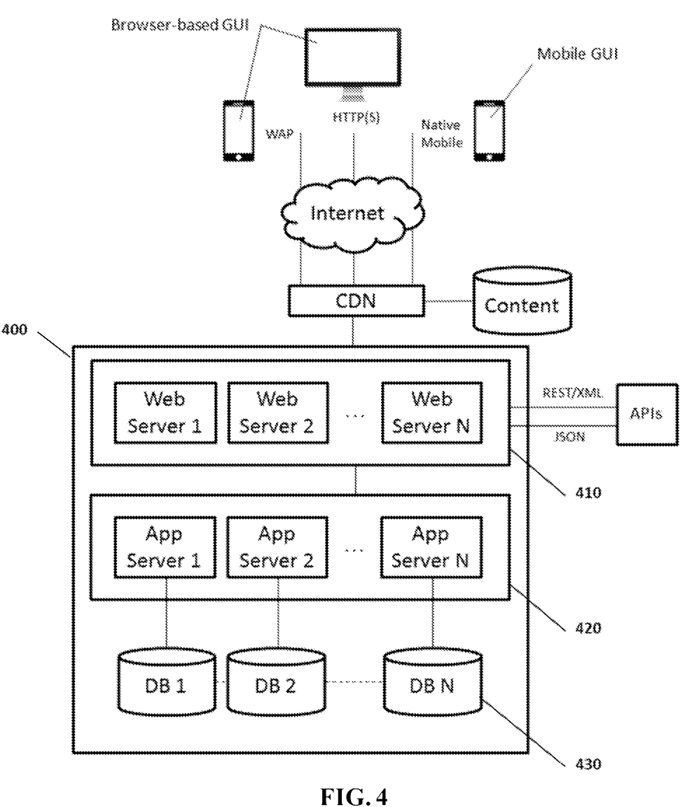

Referring to FIG. 4, in a particular embodiment, an application provision system alternatively has a distributed, cloud-based architecture 400 and comprises elastically load balanced, auto-scaling web server resources 410 and application server resources 420 as well synchronously replicated databases 430.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities that extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony®PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases or use of the same. In view of the disclosure provided herein, those of skill in the art will recognize that many databases are suitable for storage and retrieval of image information. In various embodiments, suitable databases include, by way of non-limiting examples, relational databases, non-relational databases, object oriented databases, object databases, entity-relationship model databases, associative databases, XML databases, and document oriented databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### EXAMPLES

The following illustrative examples are representative of embodiments of the software applications, systems, and methods described herein and are not meant to be limiting in any way.

### Example 1- Workflow for Whole Slide Imaging to Detect and Quantify Extrachromosomal DNA (ecDNA)

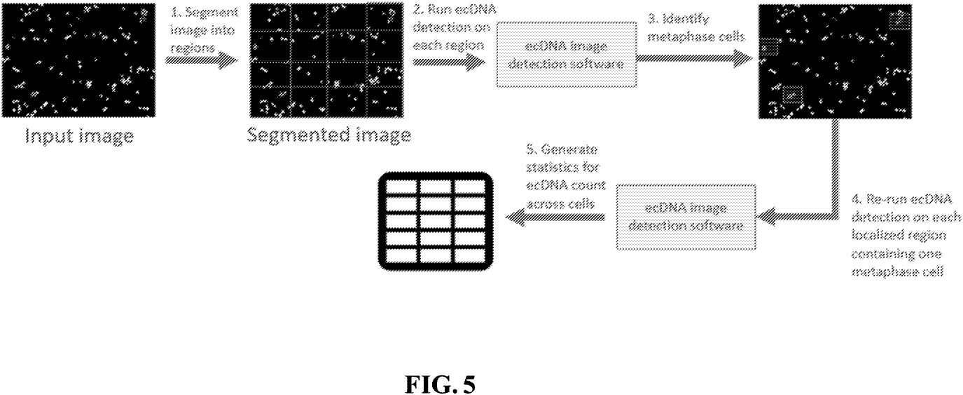

FIG. 5 shows an example workflow for whole slide imaging analysis for quantitation of ecDNA in metaphase cells. A whole-slide image comprising cells stained with a nucleic acid stain (e.g., 4′, 6-diamidino-2-phenylindole, “DAPI” or Hoechst) is used as an input image. The whole slide image (see e.g., FIG. 6) contains cells with different phases in the cell cycle, and it is useful to identify features of interest (e.g., ecDNA and the chromosomal signatures) in metaphase or interphase cells. The image is partitioned into individual first regions (see, e.g., FIG. 7). The individual first regions are segmented and subjected to an image recognition algorithm, which recognizes a first set of features (e.g., ecDNA and the chromosomal signatures) in each of the individual first regions.

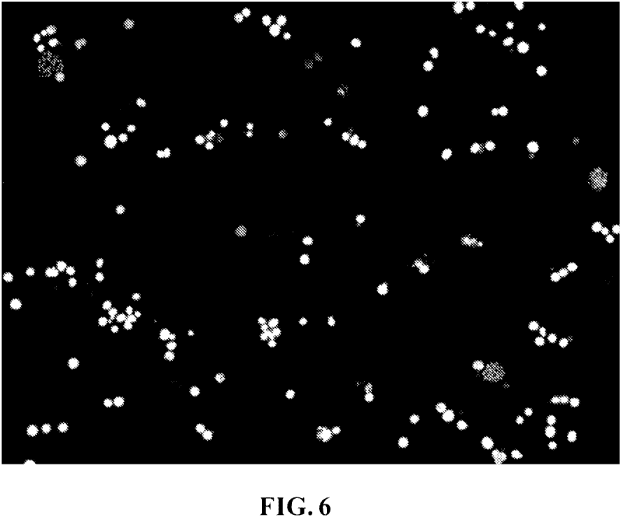

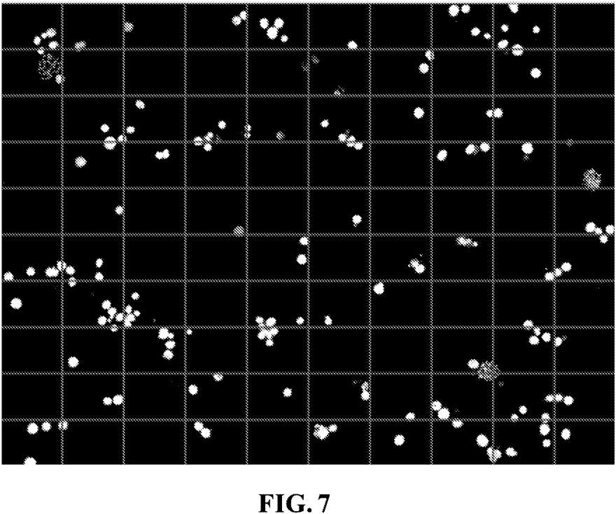

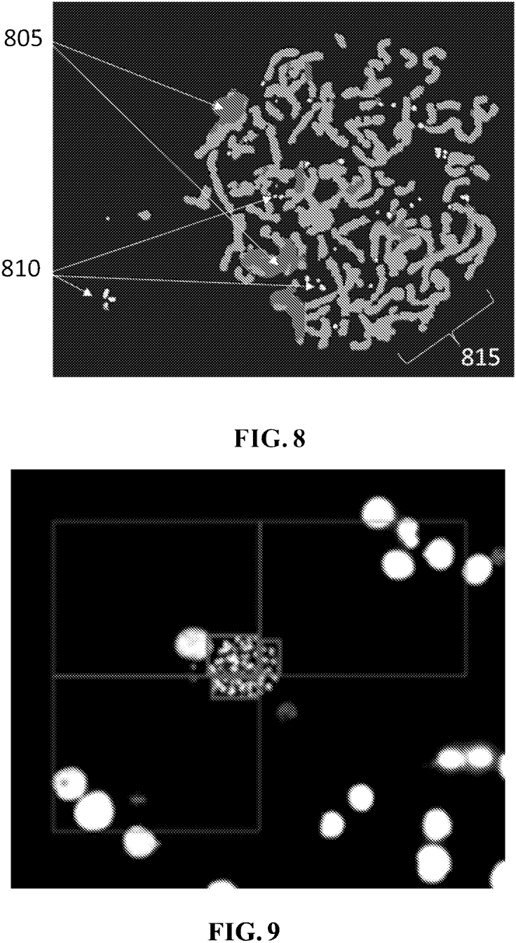

FIG. 8 shows an example of an output from the segmentation and image recognition algorithm in one of the individual first regions. The individual first region comprises a cell and has marked or identified the nuclei 805, the ecDNA 810, and the chromosomes 815.

In some instances, the individual first regions contain multiple cells in metaphase, or a cell in metaphase spans across two or more of the individual regions. To identify the boundaries of the metaphase cells, the chromosome locations are modeled using a statistical operation, e.g., density-based spatial clustering of applications with noise (DBSCAN) to determine the number of cells that are in metaphase in the image. In such an example, the boundaries of the distributions are marked in the image (see e.g., FIG. 9), and the overlapping clusters are merged to generate a set of second regions, which second regions each comprise a single cell (see, e.g.,

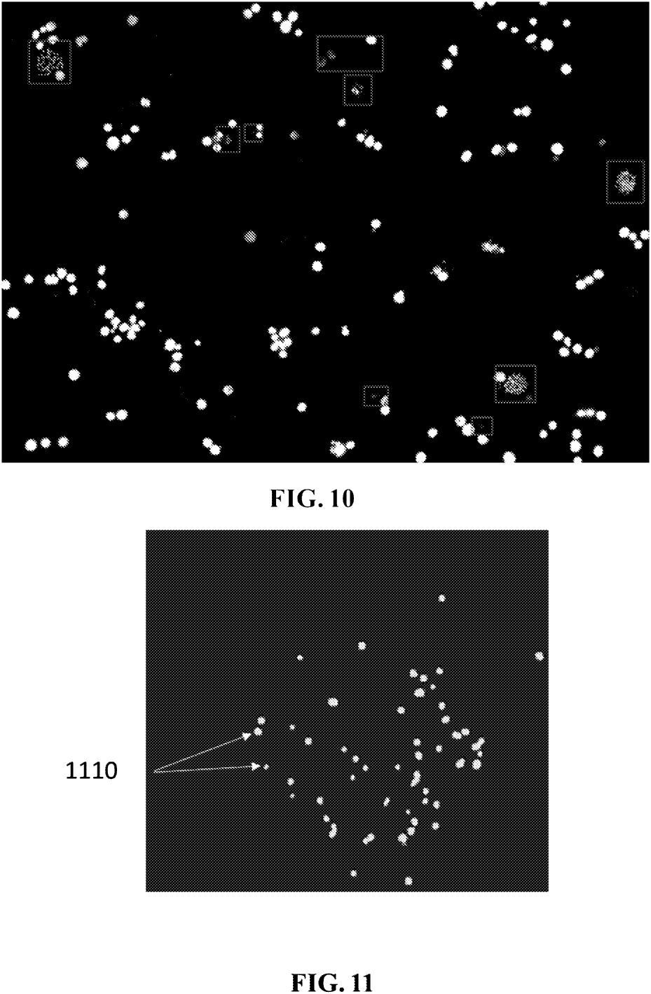

FIG. 10). The second regions are then subjected to another image segmentation and recognition algorithm, which recognizes the number of ecDNA per cell in each of the second regions. The number of ecDNA per metaphase cell are quantitated across the entire image. In some instances, additional processing is implemented (e.g., via the one or more processors). For instance, the ecDNA to chromosomal signal or locations are compared in each of the second regions, and any outliers (e.g., regions with high ecDNA signal but low or no chromosomal signal) are removed. For example, FIG. 11 shows an image of a second region comprising outliers 1110 which appear like ecDNA. However, as no chromosomal signature is present, the outliers 1110 are marked as an outlier and removed from further quantitation or analysis.

The ecDNA may be quantitated, and other properties of the ecDNA may be electronically output in a report. In certain examples, the report comprises other signatures or statistics of the image (e.g., average number of ecDNA per cell, spatial locations of ecDNA relative to chromosomal DNA, location of outliers, etc.). In other examples, the report comprises a text file.

### Example 2 - Workflow for Whole Slide Imaging to Detect and Quantify Extrachromosomal

### DNA (ecDNA) with Downsampling

FIG. 1B shows an example workflow for whole slide imaging analysis for quantitation of ecDNA in metaphase cells. A whole-slide image comprising cells stained with a nucleic acid stain (e.g., 4′, 6-diamidino-2-phenylindole, “DAPI” or Hoechst), or a multi-color image (e.g., FISH) of a whole slide is used as an input image. The whole slide image contains cells with different phases in the cell cycle, and it is useful to identify features of interest (e.g., ecDNA and the chromosomal signatures) in metaphase or interphase cells. The image is first downsampled (see, e.g., FIG. 12) to reduce the resolution of the image (e.g., by approximately 90%). For multi-color images, the image is converted to grayscale (e.g., convert multichannel image into images for each single channel image) before down-sampling.

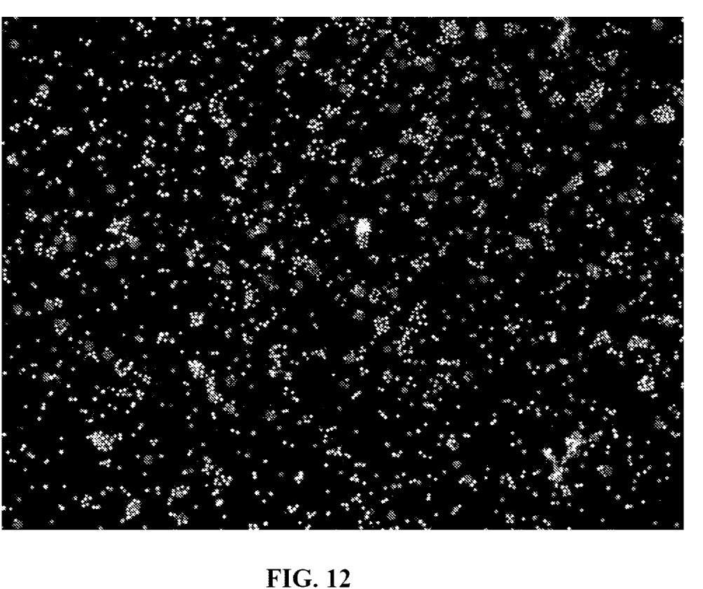

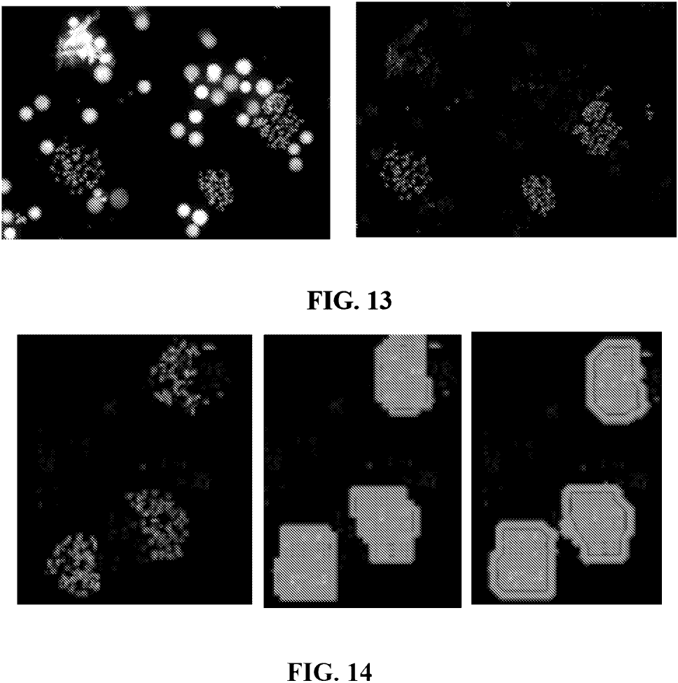

The down-sampled image is segmented using at least one processor. In some instances, the segmentation comprises white top-hat filtering. The white top-hat filtering process performs at least one morphological opening, which comprises performing an erosion and dilation to remove small, high-intensity features (e.g., chromosomes, ecDNA, noise), leaving larger, high pixel intensity nuclei. The compact nuclei (or interphase nuclei) are identified and removed from the down-sampled image (i.e., the morphological opening is subtracted from the image). FIG. 13 shows example images resulting from the white top-hat filtering process. The left panel shows a section of the down-sampled image and the right panel shows the same section of the down-sampled image after white top-hat filtering, which removes the compact nuclei along with other debris, noise, etc. The resultant image is free of compact nuclei.

Further processing of the compact-nuclei-free image is performed, using the one or more processors. In some instances, such processing includes identification of regions of high pixel intensity (e.g., which represent or are indicative of metaphase spreads). In one example, a window (e.g., kernel size of 16 pixels by 16 pixels) slides across the compact-nuclei-free image. At each location (pixel) in the image, the pixel values in the window are summed to generate a total intensity value of the window for each location (pixel). If the total intensity of a window is above a threshold value (e.g., a user-set threshold value, or a value output by one of the machine learning algorithms described herein), the window is marked by the at least one processor as potentially containing a part of a metaphase image. FIG. 14 shows an example of a compact-nuclei-free image (left), and the image having a plurality of windows (e.g., regions) that have a summed pixel intensity value above the threshold (center).

The one or more processors then generates a plurality of contours around at least a subset of the windows (regions) (see FIG. 14, right panel). The contours each comprise or surround a plurality of overlapping windows (regions) that have a summed intensity value greater than the threshold value. The coordinates of the centers of each contour (e.g., centroid) is obtained for each contour.

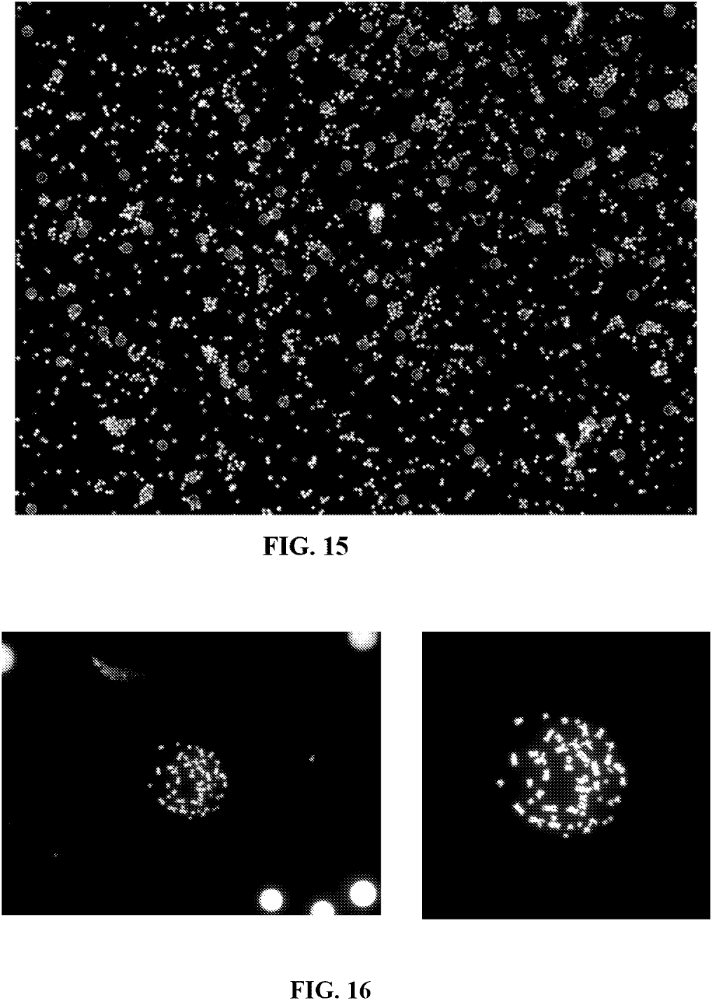

Using the coordinates of the contours, the at least one processor then partitions the original (high-resolution) image into a plurality of second images or regions. For example, FIG. 15 shows an example in which the coordinates from the contours of the down-sampled, compact-nuclei-free image is mapped to the original, high-resolution image. Each circle in the original, high-resolution image corresponds to a single contour identified in the down-sampled image. Each image or region of the plurality of second images or regions comprises a single metaphase spread. If separate images are generated, each image comprises a single metaphase spread, located in the center of the image. FIG. 16 compares an example of a region of a whole-slide that is manually captured (left) and one that is identified using the computer-implemented method (right), in which the metaphase spread is located centrally in the image.

The second images or regions are then subjected to another image segmentation and recognition algorithm, which recognizes the number of ecDNA per cell or region in each of the second images or regions. The number of ecDNA per metaphase cell are quantitated across the entire whole-slide image. In some instances, additional processing is implemented (e.g., via the one or more processors). For instance, the ecDNA to chromosomal signal or locations are compared in each of the second regions, and any outliers (e.g., regions with high ecDNA signal but low or no chromosomal signal) are removed, as described above.

The ecDNA may be quantitated, and other properties of the ecDNA may be electronically output in a report. In certain examples, the report comprises other signatures or statistics of the image (e.g., average number of ecDNA per cell, spatial locations of ecDNA relative to chromosomal DNA, location of outliers, etc.). In other examples, the report comprises a text file.

Such examples of whole-slide imaging and quantitation is useful in reducing bias when quantitating the number of ecDNA present per cell by removing manual surveying of slides and image collection. Rather, in such examples, the whole slide is imaged and analyzed using one or more automated methods.

### Example 3 -Automated Detection and Quantitation of Extrachromosomal DNA (ecDNA) Improves Accuracy

The computer-implemented methods are validated for the ability to identify and quantify the presence of ecDNA using two different cell lines: COLO320DM and H2170. Cells are treated for several hours with colcemid to arrest dividing cells in metaphase. The cells are collected using trypsin, washed with PBS, and incubated in a hypotonic solution. Cells are then immediately fixed in suspension using Carnoy’s fixative. Samples are dropped on humidified slides and air dried. ProLong Gold Antifade Mountant with DAPI is added to each slide prior to the addition of coverslips. Images are captured using a 60x oil objective (BZ-X800 fluorescent microscope, Keyence).

A ground truth is established by having an experienced researcher identify and count the number of metaphase nuclei in a whole slide images of DAPI stained COLO320DM cells and DAPI stained H2170 cells. In one example, the researcher identified 41 metaphase spreads, by eye, in each of the COLO320DM whole slide and the H2170 whole slide image. The computer-implemented process (e.g., FIG. 1B) identified 92 metaphase spreads in the COLO320DM line, which included all 41 of the metaphase spreads detected by the researcher. On the H2170 line, the software detected 55 metaphases, which included all 41 of the metaphases detected by the researcher. Visual inspection of the additional metaphases detected by the automated approach revealed that these extra images were indeed metaphase spreads, illustrating the utility of the automated approach in identifying metaphase spreads.

In order to ensure that downstream analyses using the automated approach gives comparable results to manual imaging, 15 metaphase spreads from COLO320DM and H2170 were manually imaged by an experienced researcher. These images were compared directly to the same 15 metaphase spreads that were identified from the automated approach. An automated deep learning ecDNA counting algorithm was applied to both sets.

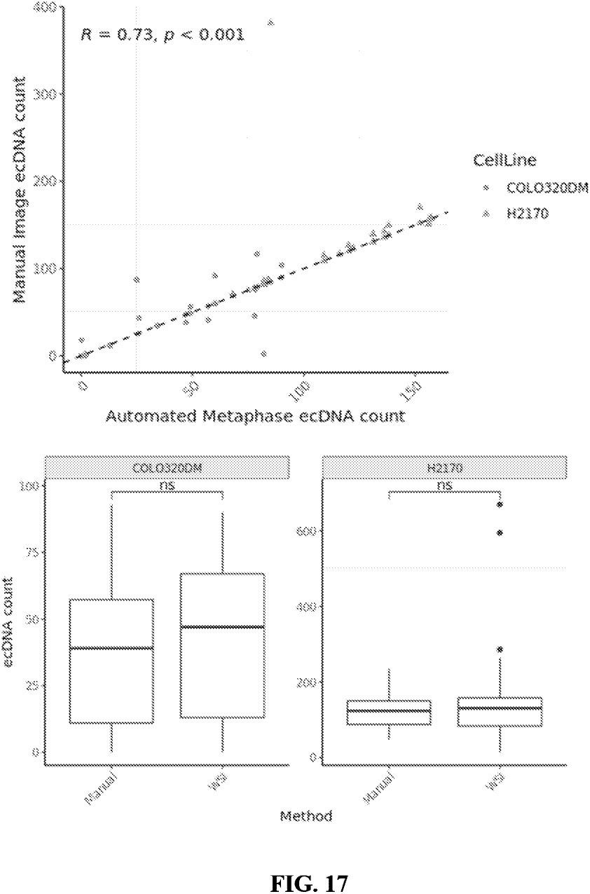

Downstream analyses of using a deep learning algorithm for counting ecDNA in the 15 manually captured metaphase images versus the same metaphases identified by whole slide imaging (WSI) reveal comparable ecDNA counts (FIG. 17, top), outside of outliers due to the computer-implemented method counting debris as ecDNA. These issues are related to downstream analyses (e.g., segmentation and quantitation of ecDNA) and not due to the automated capture pipeline (e.g., down-sampling, removing compact nuclei, generating contours, and partitioning of the original image using the coordinates of the contours, as is described herein).

Comparing the distribution of ecDNA in the 15 manually captured metaphase images (left hand column of each plot in FIG. 17, bottom) versus all metaphases detected by the automated process (the right column of each plot of FIG. 17, bottom) reveal that no statistically significant difference in ecDNA distribution is detected.

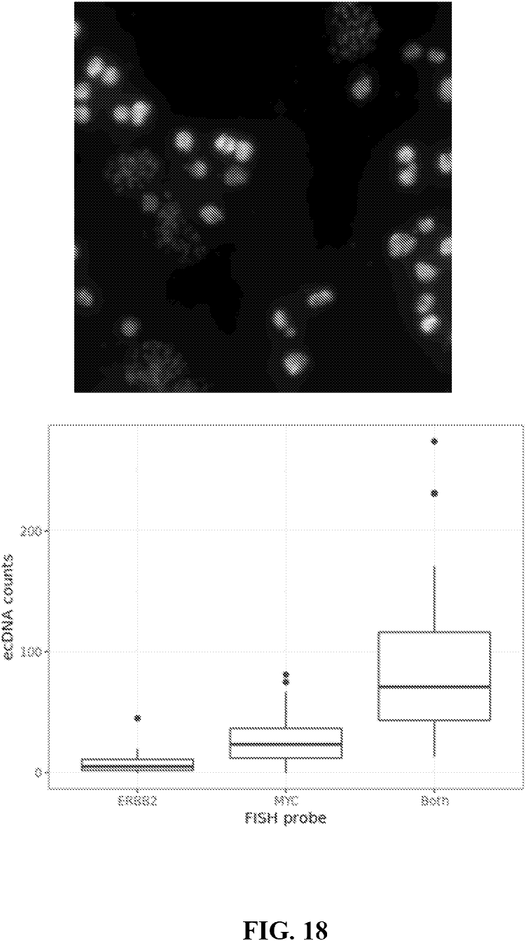

This approach may be applied to WSI FISH to reveal aspects that would generally not be available by other detection techniques. For example, qtPCR of H2170 reveals that the cell line contains both amplified MYC and ERBB2 (data not shown), but it does not reveal any information about their genomic locations. WSI FISH (FIG. 18, top) reveal that both MYC and ERBB2 appear on separate ecDNA, and also co-locate on the same ecDNA. Additionally, the WSI FISH demonstrate that the population having MYC and ERBB2 co-localized on ecDNA is the most dominant in the cell line (FIG. 18, bottom).

### Embodiments

In some cases, the present disclosure provides a method or system according to the following embodiments:
1. A computer-implemented method of eliminating bias in detecting nucleic acids present in a plurality of cells in a first image, said computer-implemented method comprising:
   (a) down-sampling, by at least one processor, said first image, thereby generating a down-sampled image;
   (b) segmenting, by said at least one processor, said down-sampled image, wherein said segmenting comprises removing, from said down-sampled image, one or more compact nuclei originating from said plurality of cells, thereby generating a compact-nuclei-free image;
   (c) automatically identifying, by said at least one processor, a plurality of first regions in said compact-nuclei-free image, wherein each region of said plurality of first regions has a summed pixel intensity value above a threshold intensity value;
   (d) generating, by said at least one processor, a plurality of contours around at least a subset of said plurality of first regions in said compact-nuclei-free image;
   (e) partitioning, by said at least one processor, said first image into a plurality of second images, using pixel locations of said plurality of contours, wherein each image of said plurality of second images comprises a single region corresponding to a single contour of said plurality of contours of said compact-nuclei-free image;
   (f) segmenting, by said at least one processor, each image of said plurality of second images to identify and/or quantify one or more nucleic acid features; and
   (g) electronically outputting information indicative of the presence or quantity of said one or more nucleic acid features present in said plurality of cells in said first image.
2. The computer-implemented method of embodiment 1, wherein said one or more nucleic acid features comprises extrachromosomal deoxyribonucleic acid (ecDNA).
3. The computer-implemented method of embodiment 1, wherein said one or more nucleic acid features comprises a chromosomal homogenous staining region (HSR).
4. The computer-implemented method of embodiment 1, wherein said one or more nucleic acid features comprises one or more gene amplifications.
5. The computer-implemented method of embodiment 1, wherein said one or more nucleic acid features comprises nuclei in metaphase.
6. The computer-implemented method of embodiment 1, wherein said information of (g) comprises one or more members selected from the group consisting of a quantity of ecDNA, a number of cells containing ecDNA, and a percentage of cells containing ecDNA.
7. The computer-implemented method of embodiment 1, wherein said information of (g) comprises a quantity of HSR on a chromosome, a quantity of HSR on ecDNA, or a ratio of pixel intensity of FISH on HSR on chromosomes to pixel intensity of FISH on ecDNA.
8. The computer-implemented method of embodiment 1, wherein said down-sampling in (a) comprises reducing a resolution of said first image or shrinking dimensions of said first image by a percentage.
9. The computer-implemented method of embodiment 8, wherein said percentage is between about 70% and about 95%.
10. The computer-implemented method of embodiment 1, wherein said segmenting in (b) comprises white top-hat filtering.
11. The computer-implemented method of embodiment 10, wherein said white top-hat filtering comprises a morphological opening, wherein said morphological opening comprises performing, using said at least one processor, one or more erosions, dilations, or a combination thereof.
12. The computer-implemented method of embodiment 1, wherein (b) comprises removing pixels belonging to said morphological opening.
13. The computer-implemented method of embodiment 1, wherein said one or more compact nuclei comprises a non-metaphase nucleus.
14. The computer-implemented method of embodiment 1, wherein (c) comprises sliding a window across said compact-nuclei-free image, wherein at each pixel location of said compact-nucleic-free image, a summation of pixel intensities in said window is performed.
15. The computer-implemented method of embodiment 14, wherein said plurality of first regions is generated from said window only if said summation of pixel intensities is greater than said threshold intensity value.
16. The computer-implemented method of embodiment 14, wherein said window has a kernel size of 16 pixels by 16 pixels.
17. The computer-implemented method of embodiment 1, wherein said pixel locations of (e) are image coordinates of centroids of said plurality of contours.
18. The computer-implemented method of embodiment 1, wherein an image of said plurality of second images comprises a single metaphase nucleus.
19. The computer-implemented method of embodiment 18, wherein said single metaphase nucleus is located in a center of said image.
20. The computer-implemented method of embodiment 1, wherein said plurality of contours comprises or surrounds overlapping first regions of said plurality of first regions.
21. The computer-implemented method of embodiment 1, wherein said one or more nucleic acid features comprises ecDNA, wherein said ecDNA comprises a first labeled probe and a second labeled probe, wherein the first and the second labeled probes each hybridize to a different feature.
22. The computer-implemented method of embodiment 21, wherein said different feature comprises a gene-specific sequence.
23. The computer-implemented method of embodiment 21, further comprising separately quantifying said ecDNA comprising said first labeled probe and said ecDNA comprising said second labeled probe.
24. The computer-implemented method of embodiment 1, wherein each contour of said plurality of contours corresponds to a cell of said plurality of cells.
25. The computer-implemented method of embodiment 1, wherein said first image comprises a plurality of images of a microscope slide comprising said plurality of cells.
26. The computer-implemented method of embodiment 25, further comprising, prior to (a), overlapping, by said at least one processor, said plurality of images to generate said first image.
27. The computer-implemented method of embodiment 26, wherein said plurality of images comprises at least 20 images.
28. The computer-implemented method of embodiment 1, wherein said one or more nucleic acid features comprises ecDNA, wherein said ecDNA comprises labeled probes.
29. The computer-implemented method of embodiment 28, wherein said labeled probes comprises gene-specific fluorescence in situ hybridization (FISH) probes.
30. The computer-implemented method of embodiment 28, wherein said labeled probes comprise colorimetric in situ hybridization (CISH) probes.
31. The computer-implemented method of embodiment 1, wherein said first image further comprises an additional plurality of cells that do not have ecDNA.
32. The computer-implemented method of embodiment 1, further comprising, performing a statistical operation on said nucleic acid features identified in (f).
33. The computer-implemented method of embodiment 32, wherein said statistical operation compares a pixel intensity and location of said nucleic acid features to a pixel intensity and location of an additional set of features of interest.
34. The computer-implemented method of embodiment 33, wherein said additional set of features of interest comprises chromosomal DNA.
35. The computer-implemented method of embodiment 34, wherein said statistical operation uses said comparison to remove outliers.
36. The computer-implemented method of embodiment 1, wherein (d) comprises using a statistical clustering algorithm of said summed pixel intensity value to generate said plurality of contours.
37. A computer-implemented system for performing non-biased, automatic detection of nucleic acids present in a plurality of cells in a first image, comprising: at least one processor configured to perform executable instructions and a memory comprising said executable instructions, which, when executed by said at least one processor, causes said at least one processor to:
   (a) down-sample said first image, thereby generating a down-sampled image;
   (b) segment said down-sampled image, wherein said segmenting comprises removing, from said down-sampled image, one or more compact nuclei originating from said plurality of cells, thereby generating a compact-nuclei-free image;
   (c) automatically identify a plurality of first regions in said compact-nuclei-free image, wherein each region of said plurality of first regions has a summed pixel intensity value above a threshold intensity value;
   (d) generate a plurality of contours around at least a subset of said plurality of first regions in said compact-nuclei-free image;
   (e) partition said first image into a plurality of second images, using pixel locations of said plurality of contours, wherein each image of said plurality of second images comprises a single region corresponding to a single contour of said plurality of contours of said compact-nuclei-free image;
   (f) segment each image of said plurality of second images to identify one or more nucleic acid features; and
   (g) electronically output information indicative of the presence or quantity of said one or more nucleic acid features present in said plurality of cells in said first image.
38. The computer-implemented system of embodiment 37, wherein said one or more nucleic acid features comprises extrachromosomal deoxyribonucleic acid (ecDNA).
39. The computer-implemented system of embodiment 37, wherein said one or more nucleic acid features comprises a chromosomal homogenous staining region (HSR).
40. The computer-implemented system of embodiment 37, wherein said one or more nucleic acid features comprises one or more gene amplifications.
41. The computer-implemented system of embodiment 37, wherein said one or more nucleic acid features comprises nuclei in metaphase.
42. The computer-implemented system of embodiment 37, wherein said information of (g) comprises one or more members selected from the group consisting of a quantity of ecDNA, a number of cells containing ecDNA, and a percentage of cells containing ecDNA.
43. The computer-implemented system of embodiment 37, wherein said information of (g) comprises a quantity of HSR on a chromosome, a quantity of HSR on ecDNA, or a ratio of pixel intensity of FISH on HSR on chromosomes to pixel intensity of FISH on ecDNA.
44. The computer-implemented system of embodiment 37, wherein said down-sampling in (a) comprises reducing a resolution of said first image or shrinking dimensions of said first image by a percentage.
45. The computer-implemented system of embodiment 44, wherein said percentage is between about 70% and about 95%.
46. The computer-implemented system of embodiment 37, wherein said segmenting in (b) comprises white top-hat filtering.
47. The computer-implemented system of embodiment 46, wherein said white top-hat filtering comprises a morphological opening, wherein said morphological opening comprises performing, using said at least one processor, one or more erosions, dilations, or a combination thereof.
48. The computer-implemented system of embodiment 37, wherein (b) comprises removing pixels belonging to said morphological opening.
49. The computer-implemented system of embodiment 37, wherein said one or more compact nuclei comprises a non-metaphase nucleus.
50. The computer-implemented system of embodiment 37, wherein (c) comprises sliding a window across said compact-nuclei-free image, wherein at each pixel location of said compact-nucleic-free image, a summation of pixel intensities in said window is performed.
51. The computer-implemented system of embodiment 50, wherein said plurality of first regions is generated from said window only if said summation of pixel intensities is greater than said threshold intensity value.
52. The computer-implemented system of embodiment 50, wherein said window has a kernel size of 16 pixels by 16 pixels.
53. The computer-implemented system of embodiment 37, wherein said pixel locations of (e) are image coordinates of centroids of said plurality of contours.
54. The computer-implemented system of embodiment 37, wherein an image of said plurality of second images comprises a single metaphase nucleus.
55. The computer-implemented system of embodiment 54, wherein said single metaphase nucleus is located in a center of said image.
56. The computer-implemented system of embodiment 37, wherein said plurality of contours comprises or surrounds overlapping first regions of said plurality of first regions.
57. The computer-implemented system of embodiment 37, wherein said one or more nucleic acid features comprises ecDNA, wherein said ecDNA comprises a first labeled probe and a second labeled probe, wherein the first and the second labeled probes each hybridize to a different feature.
58. The computer-implemented system of embodiment 57, wherein said different feature comprises a gene-specific sequence.
59. The computer-implemented system of embodiment 57, wherein said executable instructions causes said at least one processor to separately quantify said ecDNA comprising said first labeled probe and said ecDNA comprising said second labeled probe.
60. The computer-implemented system of embodiment 37, wherein each contour of said plurality of contours corresponds to a cell of said plurality of cells.
61. The computer-implemented system of embodiment 37, wherein said first image comprises a plurality of images of a microscope slide comprising said plurality of cells.
62. The computer-implemented system of embodiment 61, wherein said executable instructions causes said at least one processor to, prior to (a), overlap said plurality of images to generate said first image.
63. The computer-implemented system of embodiment 62, wherein said plurality of images comprises at least 20 images.
64. The computer-implemented system of embodiment 37, wherein said one or more nucleic acid features comprises ecDNA, wherein said ecDNA comprises labeled probes.
65. The computer-implemented system of embodiment 64, wherein said labeled probes comprises gene-specific fluorescence in situ hybridization (FISH) probes.
66. The computer-implemented system of embodiment 64, wherein said labeled probes comprise colorimetric in situ hybridization (CISH) probes.
67. The computer-implemented system of embodiment 37, wherein said first image further comprises an additional plurality of cells that do not have ecDNA.
68. The computer-implemented system of embodiment 37, wherein said executable instructions causes said at least one processor to perform a statistical operation on said nucleic acid features identified in (f).
69. The computer-implemented system of embodiment 68, wherein said statistical operation compares a pixel intensity and location of said nucleic acid features to a pixel intensity and location of an additional set of features of interest.
70. The computer-implemented system of embodiment 69, wherein said additional set of features of interest comprises chromosomal DNA.
71. The computer-implemented system of embodiment 68, wherein said statistical operation uses said comparison to remove outliers.
72. The computer-implemented system of embodiment 37, wherein (d) comprises using a statistical clustering algorithm of said summed pixel intensity value to generate said plurality of contours.
73. A non-transitory computer readable storage medium encoded with a computer program including instructions executable by a processor to perform non-biased, automatic detection of nucleic acids present in a plurality of cells in a first image, said computer program comprising:
   (a) a software module for down-sampling said first image, thereby generating a down-sampled image;
   (b) a software module for segmenting said down-sampled image, wherein said segmenting comprises removing, from said down-sampled image, one or more compact nuclei originating from said plurality of cells, thereby generating a compact-nuclei-free image;
   (c) a software module for automatically identifying a plurality of first regions in said compact-nuclei-free image, wherein each region of said plurality of first regions has a summed pixel intensity value above a threshold intensity value;
   (d) a software module for generating a plurality of contours around at least a subset of said plurality of first regions in said compact-nuclei-free image;
   (e) a software module for partitioning said first image into a plurality of second images, using pixel locations of said plurality of contours, wherein each image of said plurality of second images comprises a single region corresponding to a single contour of said plurality of contours of said compact-nuclei-free image;
   (f) a software module for segmenting each image of said plurality of second images to identify one or more nucleic acid features; and
   (g) a software module for electronically outputting information indicative of the presence or quantity of said one or more nucleic acid features present in said plurality of cells in said first image.
74. The non-transitory computer readable storage medium of embodiment 73, wherein said one or more nucleic acid features comprises extrachromosomal deoxyribonucleic acid (ecDNA).
75. The non-transitory computer readable storage medium of embodiment 73, wherein said one or more nucleic acid features comprises a chromosomal homogenous staining region (HSR).
76. The non-transitory computer readable storage medium of embodiment 73, wherein said one or more nucleic acid features comprises one or more gene amplifications.
77. The non-transitory computer readable storage medium of embodiment 73, wherein said one or more nucleic acid features comprises nuclei in metaphase.
78. The non-transitory computer readable storage medium of embodiment 73, wherein said information of (g) comprises one or more members selected from the group consisting of a quantity of ecDNA, a number of cells containing ecDNA, and a percentage of cells containing ecDNA.
79. The non-transitory computer readable storage medium of embodiment 73, wherein said information of (g) comprises a quantity of HSR on a chromosome, a quantity of HSR on ecDNA, or a ratio of pixel intensity of FISH on HSR on chromosomes to pixel intensity of FISH on ecDNA.
80. The non-transitory computer readable storage medium of embodiment 73, wherein said down-sampling in (a) comprises reducing a resolution of said first image or shrinking dimensions of said first image by a percentage.
81. The non-transitory computer readable storage medium of embodiment 80, wherein said percentage is between about 70% and about 95%.
82. The non-transitory computer readable storage medium of embodiment 73, wherein said segmenting in (b) comprises white top-hat filtering.
83. The non-transitory computer readable storage medium of embodiment 82, wherein said white top-hat filtering comprises a morphological opening, wherein said morphological opening comprises performing, using said at least one processor, one or more erosions, dilations, or a combination thereof.
84. The non-transitory computer readable storage medium of embodiment 73, wherein said segmenting in (b) comprises removing pixels belonging to said morphological opening.
85. The non-transitory computer readable storage medium of embodiment 73, wherein said one or more compact nuclei comprises a non-metaphase nucleus.
86. The non-transitory computer readable storage medium of embodiment 73, wherein said automatic identifying in (c) comprises sliding a window across said compact-nuclei-free image, wherein at each pixel location of said compact-nucleic-free image, a summation of pixel intensities in said window is performed.
87. The non-transitory computer readable storage medium of embodiment 86, wherein said plurality of first regions is generated from said window only if said summation of pixel intensities is greater than said threshold intensity value.
88. The non-transitory computer readable storage medium of embodiment 86, wherein said window has a kernel size of 16 pixels by 16 pixels.
89. The non-transitory computer readable storage medium of embodiment 73, wherein said pixel locations of (e) are image coordinates of centroids of said plurality of contours.
90. The non-transitory computer readable storage medium of embodiment 73, wherein an image of said plurality of second images comprises a single metaphase nucleus.
91. The non-transitory computer readable storage medium of embodiment 90, wherein said single metaphase nucleus is located in a center of said image.
92. The non-transitory computer readable storage medium of embodiment 73, wherein said plurality of contours comprises or surrounds overlapping first regions of said plurality of first regions.
93. The non-transitory computer readable storage medium of embodiment 73, wherein said one or more nucleic acid features comprises ecDNA, wherein said ecDNA comprises a first labeled probe and a second labeled probe, wherein the first and the second labeled probes each hybridize to a different feature.
94. The non-transitory computer readable storage medium of embodiment 93, wherein said different feature comprises a gene-specific sequence.
95. The non-transitory computer readable storage medium of embodiment 93, further comprising a software module for separately quantifying said ecDNA comprising said first labeled probe and said ecDNA comprising said second labeled probe.
96. The non-transitory computer readable storage medium of embodiment 73, wherein each contour of said plurality of contours corresponds to a cell of said plurality of cells.
97. The non-transitory computer readable storage medium of embodiment 73, wherein said first image comprises a plurality of images of a microscope slide comprising said plurality of cells.
98. The non-transitory computer readable storage medium of embodiment 97, further comprising, a software module for overlapping said plurality of images to generate said first image.
99. The non-transitory computer readable storage medium of embodiment 97, wherein said plurality of images comprises at least 20 images.
100. The non-transitory computer readable storage medium of embodiment 73, wherein said one or more nucleic acid features comprises ecDNA, wherein said ecDNA comprises labeled probes.
101. The non-transitory computer readable storage medium of embodiment 100, wherein said labeled probes comprises gene-specific fluorescence in situ hybridization (FISH) probes.
102. The non-transitory computer readable storage medium of embodiment 100, wherein said labeled probes comprise colorimetric in situ hybridization (CISH) probes.
103. The non-transitory computer readable storage medium of embodiment 73, wherein said first image further comprises an additional plurality of cells that do not have ecDNA.
104. The non-transitory computer readable storage medium of embodiment 73, further comprising, a software module for performing a statistical operation on said nucleic acid features identified in (f).
105. The non-transitory computer readable storage medium of embodiment 104, wherein said statistical operation compares a pixel intensity and location of said nucleic acid features to a pixel intensity and location of an additional set of features of interest.
106. The non-transitory computer readable storage medium of embodiment 105, wherein said additional set of features of interest comprises chromosomal DNA.
107. The non-transitory computer readable storage medium of embodiment 104, wherein said statistical operation uses said comparison to remove outliers.
108. The non-transitory computer readable storage medium of embodiment 73, wherein (d) comprises using a statistical clustering algorithm of said summed pixel intensity value to generate said plurality of contours.
109. A computer-implemented method of eliminating bias in detecting labeled nucleic acids present in a plurality of cells in a first image, said computer-implemented method comprising:
   (a) down-sampling, by at least one processor, said first image, thereby generating a down-sampled image;
   (b) segmenting, by said at least one processor, said down-sampled image, wherein said segmenting comprises removing, from said down-sampled image, one or more interphase nuclei originating from said plurality of cells, thereby generating an interphase-nuclei-free image;
   (c) automatically identifying, by said at least one processor, a plurality of first regions in said interphase-nuclei-free image, wherein each region of said plurality of first regions has a summed pixel intensity value above a threshold intensity value;
   (d) generating, by said at least one processor, a plurality of contours around at least a subset of said plurality of first regions in said interphase-nuclei-free image;
   (e) partitioning, by said at least one processor, said first image into a plurality of second images, using pixel locations of said plurality of contours, wherein each image of said plurality of second images comprises a single region corresponding to a single contour of said plurality of contours of said interphase-nuclei-free image; and
   (f) segmenting, by said at least one processor, each image of said plurality of second images to identify said labeled nucleic acids.
110. The computer-implemented method of embodiment 109, further comprising, (g) electronically outputting a report indicative of a quantity of said labeled nucleic acids present in said plurality of cells in said first image.
111. The computer-implemented method of embodiment 109, further comprising, (g) electronically outputting a report indicative of a number of cells of said plurality of cells having said labeled nucleic acids.
112. The computer-implemented method of embodiment 109, wherein said labeled nucleic acids comprise fluorescence in situ hybridization (FISH) or colorimetric in situ hybridization (CISH) probes.
113. The computer-implemented method of embodiment 109, wherein said labeled nucleic acids comprise extrachromosomal deoxyribonucleic acid (ecDNA).
114. The computer-implemented method of embodiment 109, wherein said labeled nucleic acids comprise a chromosomal homogeneous staining region (HSR).
115. The computer-implemented method of embodiment 109, wherein said one or more labeled nucleic acids comprise an amplified nucleic acid molecule.
116. A computer-implemented system for performing non-biased, automatic detection of labeled nucleic acids present in a plurality of cells in a first image, comprising: at least one processor configured to perform executable instructions and a memory comprising said executable instructions, which, when executed by said at least one processor, causes said at least one processor to:
   (a) down-sample said first image, thereby generating a down-sampled image;
   (b) segment said down-sampled image, wherein said segmenting comprises removing, from said down-sampled image, one or more interphase nuclei originating from said plurality of cells, thereby generating an interphase-nuclei-free image;
   (c) automatically identify a plurality of first regions in said interphase-nuclei-free image, wherein each region of said plurality of first regions has a summed pixel intensity value above a threshold intensity value;
   (d) generate a plurality of contours around at least a subset of said plurality of first regions in said interphase-nuclei-free image;
   (e) partition said first image into a plurality of second images, using pixel locations of said plurality of contours, wherein each image of said plurality of second images comprises a single region corresponding to a single contour of said plurality of contours of said interphase-nuclei-free image;
   (f) segment each image of said plurality of second images to identify said labeled nucleic acids; and
   (g) electronically output a report indicative of a quantity of said labeled nucleic acids present in said plurality of cells in said first image.
117. A non-transitory computer readable storage medium encoded with a computer program including instructions executable by a processor to perform non-biased, automatic detection of labeled nucleic acids present in a plurality of cells in a first image, said computer program comprising:
   (a) a software module for down-sampling said first image, thereby generating a down-sampled image;
   (b) a software module for segmenting said down-sampled image, wherein said segmenting comprises removing, from said down-sampled image, one or more interphase nuclei originating from said plurality of cells, thereby generating an interphase-nuclei-free image;
   (c) a software module for automatically identifying a plurality of first regions in said interphase-nuclei-free image, wherein each region of said plurality of first regions has a summed pixel intensity value above a threshold intensity value;
   (d) a software module for generating a plurality of contours around at least a subset of said plurality of first regions in said interphase-nuclei-free image;
   (e) a software module for partitioning said first image into a plurality of second images, using pixel locations of said plurality of contours, wherein each image of said plurality of second images comprises a single region corresponding to a single contour of said plurality of contours of said interphase-nuclei-free image;
   (f) a software module for segmenting each image of said plurality of second images to identify said labeled nucleic acids; and
   (g) a software module for electronically outputting a report indicative of a quantity of said labeled nucleic acids present in said plurality of cells in said first image.
118. A computer-implemented method of eliminating bias in a quantification of features of interest present in a plurality of cells in an image, said computer-implemented method comprising:
   (a) partitioning, by at least one processor, said image into a plurality of first regions;
   (b) segmenting, by said at least one processor, each region of said plurality of first regions to identify a first set of features, wherein said segmenting is performed using at least one pixel intensity value relative to a background intensity value;
   (c) automatically identifying, by said at least one processor, boundaries of said plurality of cells across said plurality of first regions using said at least one pixel intensity value and a pixel location of at least one feature of said first set of features;
   (d) generating, by said at least one processor, a plurality of second regions using said boundaries of said plurality of cells;
   (e) segmenting, by said at least one processor, each region of said plurality of second regions to identify said features of interest and quantify said features of interest present in a cell of said plurality of cells; and
   (f) electronically outputting a report indicative of a quantity of said features of interest present in said cell.
119. The computer-implemented method of embodiment 118, wherein said image comprises a plurality of images of a microscope slide comprising said plurality of cells.
120. The computer-implemented method of embodiment 119, further comprising, prior to (a), overlapping, by said at least one processor, said plurality of images to generate said image.
121. The computer-implemented method of embodiment 120, wherein said plurality of images comprises at least 20 images.
122. The computer-implemented method of embodiment 118, wherein said first set of features or said features of interest comprises non-chromosomal DNA.
123. The computer-implemented method of embodiment 122, wherein said non-chromosomal DNA is extrachromosomal DNA (ecDNA).
124. The computer-implemented method of embodiment 122, wherein said first set of features or said features of interest further comprises chromosomal DNA.
125. The computer-implemented method of embodiment 122, wherein said first set of features or said features of interest further comprises fluorescently labeled probes.
126. The computer-implemented method of embodiment 125, wherein said fluorescently labeled probes comprises gene-specific fluorescence in situ hybridization (FISH) probes.
127. The computer-implemented method of embodiment 118, wherein said first set of features or said features of interest comprise gene-specific labeled probes.
128. The computer-implemented method of embodiment 127, wherein said labeled probes comprise FISH probes or colorimetric in situ hybridization (CISH) probes.
129. The computer-implemented method of embodiment 118, wherein said image further comprises an additional plurality of cells that do not comprise said features of interest.
130. The computer-implemented method of embodiment 118, further comprising, performing a statistical operation on said features of interest identified in (e).
131. The computer-implemented method of embodiment 130, wherein said statistical operation compares a pixel intensity and location of a subset of said features of interest to a pixel intensity and location of an additional subset of said features of interest.
132. The computer-implemented method of embodiment 131, wherein said subset of said features of interest comprises ecDNA and said additional subset of said features of interest comprises chromosomal DNA.
133. The computer-implemented method of embodiment 131, wherein said statistical operation uses said comparison to remove outliers.
134. The computer-implemented method of embodiment 118, wherein said cell is a metaphase cell.
135. The computer-implemented method of embodiment 118, wherein said cell is an interphase cell.
136. The computer-implemented method of embodiment 118, wherein (c) comprises using a statistical clustering algorithm of said at least one pixel intensity value to identify said boundaries of said plurality of cells.
137. The computer-implemented method of embodiment 136, wherein said statistical clustering algorithm is density-based spatial clustering of applications with noise (DBSCAN).
138. The computer-implemented method of embodiment 118, wherein (d) comprises overlapping a cluster of said boundaries of said plurality of cells to generate said plurality of second regions.
139. The computer-implemented method of embodiment 118, wherein each of said plurality of second regions has a single cell.
140. A computer-implemented system for performing non-biased, automatic quantification of features of interest present in a plurality of cells in an image, comprising: at least one processor configured to perform executable instructions and a memory comprising said executable instructions, which, when executed by said at least one processor, causes said at least one processor to:
   (a) partition said image into a plurality of first regions;
   (b) segment each of said plurality of first regions to identify a first set of features, wherein said segmenting is performed using at least one pixel intensity value relative to a background intensity value;
   (c) identify boundaries of said plurality of cells across said plurality of first regions using said at least one pixel intensity value and a pixel location of at least one feature of said first set of features;
   (d) generate a plurality of second regions using said boundaries of said plurality of cells;
   (e) segment each of said plurality of second regions to identify said features of interest and quantify said features of interest present in a cell of said plurality of cells; and
   (f) electronically output a report indicative of a quantity of said features of interest present in said cell.
141. The computer-implemented system of embodiment 140, wherein said image comprises a plurality of images of a microscope slide comprising said plurality of cells.
142. The computer-implemented system of embodiment 141, wherein said executable instructions cause said at least one processor to, prior to (a), overlap said plurality of images to generate said image.
143. The computer-implemented system of embodiment 142, wherein said plurality of images comprises at least 20 images.
144. The computer-implemented system of embodiment 140, wherein said first set of features or said features of interest comprises non-chromosomal DNA.
145. The computer-implemented system of embodiment 144, wherein said non-chromosomal DNA is extrachromosomal DNA (ecDNA).
146. The computer-implemented system of embodiment 144, wherein said first set of features or said features of interest further comprises chromosomal DNA.
147. The computer-implemented system of embodiment 144, wherein said first set of features or said features of interest further comprises fluorescently labeled probes.
148. The computer-implemented system of embodiment 147, wherein said fluorescently labeled probes comprises gene-specific fluorescence in situ hybridization (FISH) probes.
149. The computer-implemented system of embodiment 140, wherein said first set of features or said features of interest comprise gene-specific labeled probes.
150. The computer-implemented system of embodiment 149, wherein said labeled probes comprise FISH probes or colorimetric in situ hybridization (CISH) probes.
151. The computer-implemented system of embodiment 140, wherein said image further comprises an additional plurality of cells that do not comprise said features of interest.
152. The computer-implemented system of embodiment 140, wherein said executable instructions cause said at least one processor to perform a statistical operation on said features of interest identified in (e).
153. The computer-implemented system of embodiment 152, wherein said statistical operation compares a pixel intensity and location of a subset of said features of interest to a pixel intensity and location of an additional subset of said features of interest.
154. The computer-implemented system of embodiment 153, wherein said subset of said features of interest comprises ecDNA and said additional subset of said features of interest comprises chromosomal DNA.
155. The computer-implemented system of embodiment 153, wherein said statistical operation uses said comparison to remove outliers.
156. The computer-implemented system of embodiment 140, wherein said cell is a metaphase cell.
157. The computer-implemented system of embodiment 140, wherein said cell is an interphase cell.
158. The computer-implemented system of embodiment 140, wherein (c) comprises using a statistical clustering of said at least one pixel intensity value to identify said boundaries of said plurality of cells.
159. The computer-implemented system of embodiment 158, wherein said statistical clustering is density-based spatial clustering of applications with noise (DBSCAN).
160. The computer-implemented system of embodiment 140, wherein (d) comprises overlapping a cluster of said boundaries of said plurality of cells to generate said plurality of second regions.
161. The computer-implemented system of embodiment 140, wherein each of said plurality of second regions has a single cell.
162. A non-transitory computer readable storage medium encoded with a computer program including instructions executable by a processor to perform non-biased, automatic quantification of features of interest present in a plurality of cells in an image, said computer program comprising:
   (a) a software module for partitioning said image into a plurality of first regions;
   (b) a software module for segmenting each of said plurality of first regions to identify a first set of features, wherein said segmenting is performed using at least one pixel intensity value relative to a background intensity value;
   (c) a software module for automatically identifying boundaries of said plurality of cells across said plurality of first regions using said at least one pixel intensity value and a pixel location of at least one feature of said first set of features;
   (d) a software module for generating a plurality of second regions using said boundaries of said plurality of cells;
   (e) a software module for segmenting each of said plurality of second regions to identify said features of interest and quantify said features of interest present in a cell of said plurality of cells; and
   (f) a software module for electronically outputting a report indicative of a quantity of said features of interest present in said cell.
163. The non-transitory computer readable storage medium of embodiment 162, wherein said image comprises a plurality of images of a microscope slide comprising said plurality of cells.
164. The non-transitory computer readable storage medium of embodiment 163, wherein said computer program further comprises a software module for overlapping said plurality of images to generate said image.
165. The non-transitory computer readable storage medium of embodiment 164, wherein said plurality of images comprises at least 20 images.
166. The non-transitory computer readable storage medium of embodiment 162, wherein said first set of features or said features of interest comprises non-chromosomal DNA.
167. The non-transitory computer readable storage medium of embodiment 166, wherein said non-chromosomal DNA is extrachromosomal DNA (ecDNA).
168. The non-transitory computer readable storage medium of embodiment 166, wherein said first set of features or said features of interest further comprises chromosomal DNA.
169. The non-transitory computer readable storage medium of embodiment 166, wherein said first set of features or said features of interest further comprises fluorescently labeled probes.
170. The non-transitory computer readable storage medium of embodiment 169, wherein said fluorescently labeled probes comprises gene-specific fluorescence in situ hybridization (FISH) probes.
171. The non-transitory computer readable storage medium of embodiment 162, wherein said first set of features or said features of interest comprise gene-specific labeled probes.
172. The non-transitory computer readable storage medium of embodiment 171, wherein said labeled probes comprise FISH probes or colorimetric in situ hybridization (CISH) probes.
173. The non-transitory computer readable storage medium of embodiment 162, wherein said image further comprises an additional plurality of cells that do not comprise said features of interest.
174. The non-transitory computer readable storage medium of embodiment 162, wherein said computer program further comprises a software module for performing a statistical operation on said features of interest identified in (e).
175. The non-transitory computer-readable storage medium of embodiment 174, wherein said statistical operation compares a pixel intensity and location of a subset of said features of interest to a pixel intensity and location of an additional subset of said features of interest.
176. The non-transitory computer readable storage medium of embodiment 175, wherein said subset of said features of interest comprises ecDNA and said additional subset of said features of interest comprises chromosomal DNA.
177. The non-transitory computer readable storage medium of embodiment 175, wherein said statistical operation uses said comparison to remove outliers.
178. The non-transitory computer readable storage medium of embodiment 162, wherein said cell is a metaphase cell.
179. The non-transitory computer readable storage medium of embodiment 162, wherein said cell is an interphase cell.
180. The non-transitory computer-readable storage medium of embodiment 162, wherein said software module in (c) uses a statistical clustering of said at least one pixel intensity value to identify said boundaries of said plurality of cells.
181. The non-transitory computer readable storage medium of embodiment 180, wherein said statistical clustering is density-based spatial clustering of applications with noise (DBSCAN).
182. The non-transitory computer-readable storage medium of embodiment 162, wherein said software module in (d) overlaps a cluster of said boundaries of said plurality of cells to generate said plurality of second regions.
183. The non-transitory computer readable storage medium of embodiment 162, wherein each of said plurality of second regions has a single cell.
184. A computer-implemented method of eliminating bias in a quantification of features of interest present in a plurality of cells in an image, said computer-implemented method comprising:
   (a) partitioning, by at least one processor, said image into a plurality of first regions;
   (b) segmenting, by said at least one processor, each of said plurality of first regions to identify a first set of features, wherein said segmenting is performed using at least one pixel intensity value relative to a background intensity value;
   (c) automatically identifying, by said at least one processor, boundaries of said plurality of cells using said at least one pixel intensity value and a pixel location of at least one feature of said first set of features across said plurality of first regions;
   (d) segmenting, by said at least one processor, a plurality of second regions defined by said boundaries to identify said features of interest and quantify said features of interest present in a cell of said plurality of cells; and
   (e) electronically outputting a report indicative of a quantity of said features of interest present in said cell.

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

What is claimed is:

1. A computer-implemented method of eliminating bias in detecting nucleic acids present in a plurality of cells in a first image, said computer-implemented method comprising:
(a) down-sampling, by at least one processor, said first image, thereby generating a down-sampled image;
(b) segmenting, by said at least one processor, said down-sampled image, wherein said segmenting comprises removing, from said down-sampled image, one or more compact nuclei originating from said plurality of cells, thereby generating a compact-nuclei-free image;
(c) automatically identifying, by said at least one processor, a plurality of first regions in said compact-nuclei-free image, wherein each region of said plurality of first regions has a summed pixel intensity value above a threshold intensity value;
(d) generating, by said at least one processor, a plurality of contours around at least a subset of said plurality of first regions in said compact-nuclei-free image;
(e) partitioning, by said at least one processor, said first image into a plurality of second images, using pixel locations of said plurality of contours, wherein each image of said plurality of second images comprises a single region corresponding to a single contour of said plurality of contours of said compact-nuclei-free image;
(f) segmenting, by said at least one processor, each image of said plurality of second images to identify one or more nucleic acid features; and
(g) electronically outputting information indicative of the presence or quantity of said one or more nucleic acid features present in said plurality of cells in said first image.

2. The computer-implemented method of claim 1, wherein said one or more nucleic acid features comprises extrachromosomal deoxyribonucleic acid (ecDNA).

3. The computer-implemented method of claim 1 or 2, wherein said one or more nucleic acid features comprises a chromosomal homogenous staining region (HSR).

4. The computer-implemented method of any one of claims 1-3, wherein said one or more nucleic acid features comprises one or more gene amplifications.

5. The computer-implemented method of any one of claims 1-4, wherein said one or more nucleic acid features comprises nuclei in metaphase.

6. The computer-implemented method of any one of claims 1-5, wherein said information of (g) comprises one or more members selected from the group consisting of a quantity of ecDNA, a number of cells containing ecDNA, and a percentage of cells containing ecDNA.

7. The computer-implemented method of any one of claims 1-6, wherein said information of (g) comprises a quantity of HSR on a chromosome, a quantity of HSR on ecDNA, or a ratio of pixel intensity of FISH on HSR on chromosomes to pixel intensity of FISH on ecDNA.

8. The computer-implemented method of any one of claims 1-7, wherein said down-sampling in (a) comprises reducing a resolution of said first image or shrinking dimensions of said first image by a percentage.

9. The computer-implemented method of claim 8, wherein said percentage is between about 70% and about 95%.

10. The computer-implemented method of any one of claims 1-9, wherein said segmenting in (b) comprises white top-hat filtering.

11. The computer-implemented method of claim 10, wherein said white top-hat filtering comprises a morphological opening, wherein said morphological opening comprises performing, using said at least one processor, one or more erosions, dilations, or a combination thereof.

12. The computer-implemented method of any one of claims 1-11, wherein (b) comprises removing pixels belonging to said morphological opening.

13. The computer-implemented method of any one of claims 1-12, wherein said one or more compact nuclei comprises a non-metaphase nucleus.

14. The computer-implemented method of any one of claims 1-13, wherein (c) comprises sliding a window across said compact-nuclei-free image, wherein at each pixel location of said compact-nucleic-free image, a summation of pixel intensities in said window is performed.

15. The computer-implemented method of claim 14, wherein said plurality of first regions is generated from said window only if said summation of pixel intensities is greater than said threshold intensity value.

16. The computer-implemented method of claim 14 or 15, wherein said window has a kernel size of 16 pixels by 16 pixels.

17. The computer-implemented method of any one of claims 1-16, wherein said pixel locations of (e) are image coordinates of centroids of said plurality of contours.

18. The computer-implemented method of any one of claims 1-17, wherein an image of said plurality of second images comprises a single metaphase nucleus.

19. The computer-implemented method of claim 18, wherein said single metaphase nucleus is located in a center of said image.

20. The computer-implemented method of any one of claims 1-19, wherein said plurality of contours comprises or surrounds overlapping first regions of said plurality of first regions.

21. The computer-implemented method of any one of claims 1-20, wherein said one or more nucleic acid features comprises ecDNA, wherein said ecDNA comprises a first labeled probe and a second labeled probe, wherein the first and the second labeled probes each hybridize to a different feature.

22. The computer-implemented method of claim 21, wherein said different feature comprises a gene-specific sequence.

23. The computer-implemented method of claim 21 or 22, further comprising separately quantifying said ecDNA comprising said first labeled probe and said ecDNA comprising said second labeled probe.

24. The computer-implemented method of any one of claims 1-23, wherein each contour of said plurality of contours corresponds to a cell of said plurality of cells.

25. The computer-implemented method of any one of claims 1-24, wherein said first image comprises a plurality of images of a microscope slide comprising said plurality of cells.

26. The computer-implemented method of claim 25, further comprising, prior to (a), overlapping, by said at least one processor, said plurality of images to generate said first image.

27. The computer-implemented method of claim 26, wherein said plurality of images comprises at least 20 images.

28. The computer-implemented method of any one of claims 1-27, wherein said one or more nucleic acid features comprises ecDNA, wherein said ecDNA comprises labeled probes.

29. The computer-implemented method of claim 28, wherein said labeled probes comprises gene-specific fluorescence in situ hybridization (FISH) probes.

30. The computer-implemented method of claim 28, wherein said labeled probes comprise colorimetric in situ hybridization (CISH) probes.

31. The computer-implemented method of any one of claims 1-30, wherein said first image further comprises an additional plurality of cells that do not have ecDNA.

32. The computer-implemented method of any one of claims 1-31, further comprising, performing a statistical operation on said nucleic acid features identified in (f).

33. The computer-implemented method of claim 32, wherein said statistical operation compares a pixel intensity and location of said nucleic acid features to a pixel intensity and location of an additional set of features of interest.

34. The computer-implemented method of claim 33, wherein said additional set of features of interest comprises chromosomal DNA.

35. The computer-implemented method of claim 34, wherein said statistical operation uses said comparison to remove outliers.

36. The computer-implemented method of any one of claims 1-35, wherein (d) comprises using a statistical clustering algorithm of said summed pixel intensity value to generate said plurality of contours.

37. The computer-implemented method of any one of claims 1-36, wherein (f) further comprises quantifying said one or more nucleic acid features.

38. The computer-implemented method of any one of claims 1-36, wherein (f) further comprises enumerating said one or more nucleic acid features.

39. A computer-implemented system for performing non-biased, automatic detection of nucleic acids present in a plurality of cells in a first image, comprising: at least one processor configured to perform executable instructions and a memory comprising said executable instructions, which, when executed by said at least one processor, causes said at least one processor to:
(a) down-sample said first image, thereby generating a down-sampled image;
(b) segment said down-sampled image, wherein said segmenting comprises removing, from said down-sampled image, one or more compact nuclei originating from said plurality of cells, thereby generating a compact-nuclei-free image;
(c) automatically identify a plurality of first regions in said compact-nuclei-free image, wherein each region of said plurality of first regions has a summed pixel intensity value above a threshold intensity value;
(d) generate a plurality of contours around at least a subset of said plurality of first regions in said compact-nuclei-free image;
(e) partition said first image into a plurality of second images, using pixel locations of said plurality of contours, wherein each image of said plurality of second images comprises a single region corresponding to a single contour of said plurality of contours of said compact-nuclei-free image;
(f) segment each image of said plurality of second images to identify one or more nucleic acid features; and
(g) electronically output information indicative of the presence or quantity of said one or more nucleic acid features present in said plurality of cells in said first image.

40. The computer-implemented system of claim 39, wherein said one or more nucleic acid features comprises extrachromosomal deoxyribonucleic acid (ecDNA).

41. The computer-implemented system of claim 39 or 40, wherein said one or more nucleic acid features comprises a chromosomal homogenous staining region (HSR).

42. The computer-implemented system of any one of claims 39-41, wherein said one or more nucleic acid features comprises one or more gene amplifications.

43. The computer-implemented system of any one of claims 39-42, wherein said one or more nucleic acid features comprises nuclei in metaphase.

44. The computer-implemented system of any one of claims 39-43, wherein said information of (g) comprises one or more members selected from the group consisting of a quantity of ecDNA, a number of cells containing ecDNA, and a percentage of cells containing ecDNA.

45. The computer-implemented system of any one of claims 39-44, wherein said information of (g) comprises a quantity of HSR on a chromosome, a quantity of HSR on ecDNA, or a ratio of pixel intensity of FISH on HSR on chromosomes to pixel intensity of FISH on ecDNA.

46. A non-transitory computer readable storage medium encoded with a computer program including instructions executable by a processor to perform non-biased, automatic detection of nucleic acids present in a plurality of cells in a first image, said computer program comprising:
(a) a software module for down-sampling said first image, thereby generating a down-sampled image;
(b) a software module for segmenting said down-sampled image, wherein said segmenting comprises removing, from said down-sampled image, one or more compact nuclei originating from said plurality of cells, thereby generating a compact-nuclei-free image;
(c) a software module for automatically identifying a plurality of first regions in said compact-nuclei-free image, wherein each region of said plurality of first regions has a summed pixel intensity value above a threshold intensity value;
(d) a software module for generating a plurality of contours around at least a subset of said plurality of first regions in said compact-nuclei-free image;
(e) a software module for partitioning said first image into a plurality of second images, using pixel locations of said plurality of contours, wherein each image of said plurality of second images comprises a single region corresponding to a single contour of said plurality of contours of said compact-nuclei-free image;
(f) a software module for segmenting each image of said plurality of second images to identify one or more nucleic acid features; and
(g) a software module for electronically outputting information indicative of the presence or quantity of said one or more nucleic acid features present in said plurality of cells in said first image.

47. The non-transitory computer readable storage medium of claim 46, wherein said one or more nucleic acid features comprises extrachromosomal deoxyribonucleic acid (ecDNA).

48. The non-transitory computer readable storage medium of claim 46 or 47, wherein said one or more nucleic acid features comprises a chromosomal homogenous staining region (HSR).

49. The non-transitory computer readable storage medium of any one of claims 46-48, wherein said one or more nucleic acid features comprises one or more gene amplifications.

50. The non-transitory computer readable storage medium of any one of claims 46-49, wherein said one or more nucleic acid features comprises nuclei in metaphase.

51. The non-transitory computer readable storage medium of any one of claims 46-50, wherein said information of (g) comprises one or more members selected from the group consisting of a quantity of ecDNA, a number of cells containing ecDNA, and a percentage of cells containing ecDNA.

52. The non-transitory computer readable storage medium of any one of claims 46-51, wherein said information of (g) comprises a quantity of HSR on a chromosome, a quantity of HSR on ecDNA, or a ratio of pixel intensity of FISH on HSR on chromosomes to pixel intensity of FISH on ecDNA.
